SEQUENCE LISTING (4 sequences)

SEQ ID NO: 1 - DNA, 1602 bp, Artificial sequence
  Synthetic polynucleotide
atggggccaagaggagctgcatcattacccaggggaccagggcccaggagactgctgctgcctgtggtgctgcccctgttattactgctgctgctggctcctcctggcagcggagctggagcctcccgccctcctcacctggtgttcctgctggccgacgatctgggctggaacgacgtgggcttccacggcagcaggatcagaacccctcacctggatgccctggctgctggaggcgtgctgctggacaattactacacccagcctctgtgcaccccctctaggagccagctgctgaccggcaggtaccagatcagaaccggcctgcagcaccagatcatctggccttgccagccttcctgcgtgcctctggacgagaagctgctgcctcagctgctgaaggaggctggctacaccacccacatggtgggcaagtggcacctgggcatgtacaggaaggagtgtctgcctacccggaggggattcgatacctacttcggctacctgctgggctctgaggactactacagccacgagaggtgcaccctgatcgatgccctgaacgtgaccagatgtgccctggatttccgggacggagaggaggtggctaccggatacaagaacatgtactccaccaatatcttcaccaagagagccatcgccctgatcaccaatcacccccctgagaagcctctgttcctgtacctggccctgcagtctgtgcacgagcctctgcaggtgcccgaggagtacctgaagccctacgatttcatccaggacaagaaccggcaccactacgccggcatggtgagcctgatggacgaggctgtgggaaatgtgaccgccgccctgaagagctccggactgtggaacaataccgtgttcatcttctccaccgataacggaggacagaccctggctggaggaaacaattggcctctgaggggaaggaagtggtccctgtgggagggaggcgtgagaggagtgggattcgtggcttctcctctgctgaagcagaagggcgtgaagaacagagagctgatccacatcagcgactggctgcctaccctggtgaagctggccaggggccacaccaatggcaccaagcccctggatggcttcgacgtgtggaagaccatctctgagggctccccttctccccgcatcgagctgctgcacaacatcgatcccaatttcgtggactctagcccttgccccaggaacagcatggcccctgccaaggacgattcctctctgcccgagtactccgccttcaacacctctgtgcacgctgctatcaggcacggcaattggaagctgctgaccggctaccctggctgtggctactggttcccccctcccagccagtacaacgtgagcgagatccccagctccgaccctcccaccaagaccctgtggctgttcgacatcgatcgggaccctgaggagcgccacgatctgtccagggagtacccccacatcgtgaccaagctgctgagcaggctgcagttctaccacaagcacagcgtgcccgtgtacttccccgcccaggacccccggtgcgaccccaaggccaccggcgtgtggggcccctggatgtga

SEQ ID NO: 2 - DNA, 1602 bp, Artificial sequence
  Synthetic polynucleotide
atggggcctagaggggctgctagcctgcctagaggccctggacctagaaggctgctgctgcctgtggtgcttcctctcctgctgcttcttttactggccccccctggctctggggctggggctagcagacccccccatttggtgttcctgctggctgatgacctgggctggaatgatgtgggctttcatggcagcagaatcagaaccccccatcttgatgccctggctgctgggggggtgctgctggacaactactacacacagcccctgtgcacccctagcagaagccaactcctgactggcagatatcagatcagaactggcctgcagcatcagatcatctggccctgtcagcctagctgtgtgcccctggatgagaagctgctgcctcagctgctgaaggaggctggctacaccacccacatggtgggcaagtggcacctgggcatgtacagaaaggagtgcctgcccacaagaagaggctttgacacctactttggctacctgctgggctctgaggactactacagccatgagagatgcaccctgattgatgccctgaatgtgacaagatgtgccctggacttcagagatggggaagaggtggccactggctacaagaacatgtacagcaccaacatcttcaccaagagagccattgccctgatcaccaaccatccccctgaaaagcccctgttcctgtacctggccctgcagtctgtgcatgagcccctgcaagtgcctgaggagtacctgaagccctatgacttcatccaagacaagaacagacaccactatgctggcatggtgagcctgatggatgaggctgtgggcaatgtgacagctgccctgaagagctctggcctgtggaacaacacagtgttcatcttcagcacagacaatgggggccaaaccctggctgggggcaataattggcccctgagaggcagaaagtggagcctgtgggaggggggggtgagaggggtgggctttgtggctagccccctgctgaagcagaagggggtgaagaacagagagctgatccacatctctgactggctgcccaccctggtgaagctggctagaggccacaccaatggcaccaagcccctggatggctttgatgtgtggaagaccatctctgagggcagccctagccctagaattgagctgctgcacaacattgaccccaactttgtggacagcagcccctgccctagaaacagcatggcccctgccaaggatgacagcagcctgcctgagtactctgccttcaacacctctgtgcacgctgccatcagacacggcaactggaagctgctgactggctaccctggctgtggctactggttcccccccccttctcagtacaatgtgtctgagatccctagctctgacccccccaccaaaaccctgtggctgtttgacattgacagagaccctgaggagagacacgacctgagcagagagtacccccacattgtgaccaagctgctgagcagactgcagttctaccacaagcactctgtgcctgtgtacttccctgcccaagaccctagatgtgaccccaaggccactggggtgtggggcccctggatgtga

SEQ ID NO: 3 - DNA, 1602 bp, Artificial sequence
  Synthetic polynucleotide
  misc_feature (9)..(9): n is a, c, g, or t
  misc_feature (15)..(15): n is a, c, g, or t
  misc_feature (21)..(21): n is a, c, g, or t
  misc_feature (39)..(39): n is a, c, g, or t
  misc_feature (72)..(72): n is a, c, g, or t
  misc_feature (75)..(75): n is a, c, g, or t
  misc_feature (99)..(99): n is a, c, g, or t
  misc_feature (102)..(102): n is a, c, g, or t
  misc_feature (111)..(111): n is a, c, g, or t
  misc_feature (114)..(114): n is a, c, g, or t
  misc_feature (129)..(129): n is a, c, g, or t
  misc_feature (132)..(132): n is a, c, g, or t
  misc_feature (156)..(156): n is a, c, g, or t
  misc_feature (198)..(198): n is a, c, g, or t
  misc_feature (210)..(210): n is a, c, g, or t
  misc_feature (234)..(234): n is a, c, g, or t
  misc_feature (261)..(261): n is a, c, g, or t
  misc_feature (267)..(267): n is a, c, g, or t
  misc_feature (279)..(279): n is a, c, g, or t
  misc_feature (282)..(282): n is a, c, g, or t
  misc_feature (306)..(306): n is a, c, g, or t
  misc_feature (321)..(321): n is a, c, g, or t
  misc_feature (471)..(471): n is a, c, g, or t
  misc_feature (477)..(477): n is a, c, g, or t
  misc_feature (540)..(540): n is a, c, g, or t
  misc_feature (597)..(597): n is a, c, g, or t
  misc_feature (609)..(609): n is a, c, g, or t
  misc_feature (612)..(612): n is a, c, g, or t
  misc_feature (660)..(660): n is a, c, g, or t
  misc_feature (801)..(801): n is a, c, g, or t
  misc_feature (843)..(843): n is a, c, g, or t
  misc_feature (846)..(846): n is a, c, g, or t
  misc_feature (864)..(864): n is a, c, g, or t
  misc_feature (879)..(879): n is a, c, g, or t
  misc_feature (909)..(909): n is a, c, g, or t
  misc_feature (927)..(927): n is a, c, g, or t
  misc_feature (1065)..(1065): n is a, c, g, or t
  misc_feature (1212)..(1212): n is a, c, g, or t
  misc_feature (1260)..(1260): n is a, c, g, or t
  misc_feature (1269)..(1269): n is a, c, g, or t
  misc_feature (1296)..(1296): n is a, c, g, or t
  misc_feature (1326)..(1326): n is a, c, g, or t
  misc_feature (1359)..(1359): n is a, c, g, or t
  misc_feature (1362)..(1362): n is a, c, g, or t
  misc_feature (1395)..(1395): n is a, c, g, or t
  misc_feature (1401)..(1401): n is a, c, g, or t
  misc_feature (1503)..(1503): n is a, c, g, or t
  misc_feature (1527)..(1527): n is a, c, g, or t
  misc_feature (1560)..(1560): n is a, c, g, or t
atggggccnagaggngctgcnagcttgcccagaggcccnggacctaggaggctgctgctgcctgtggtgctnccnctgctgctgctgctgttgctggcnccncctggctcnggngctggggccagccgnccnccccacctggtgttcctgctggcngacgacctgggctggaacgacgtgggcttccacggcagcagnatcagaaccccncacctggatgccctggctgctggnggggtgctgctggacaactactacacncagccnctgtgcaccccntcnaggagccagctgctgactggcagntaccagatcagaacnggcctgcagcaccagatcatctggccctgtcagcctagctgtgtgcctctggatgagaagctgctgcctcagctgctgaaggaggctggctacaccacccacatggtgggcaagtggcacctgggcatgtacaggaaggagtgcctgccnacccgnagaggatttgatacctactttggctacctgctgggctctgaggactactacagccatgagagntgcaccctgattgatgccctgaatgtgacaagatgtgccctggatttccgagatggngaagaggtggcnacnggatacaagaacatgtactccaccaacatcttcaccaagagagcgatngccctgatcaccaaccatccccctgagaagcctctgttcctgtacctggccctgcatgctgtgcatgagcccctgcaggtgcctgaggagtacctgaagccctatgacttcatccaagacaagaacaggcaccactatgcnggcatggtgagcctgatggatgaggctgtgggaaatgtgacngcngccctgaagagctctggnctgtggaacaacacngtgttcatcttctccacagataacggaggncagaccctggctgggggnaataattggcccctgagaggaagaaagtggagcctgtgggagggaggcgtgagaggggtgggctttgtggctagccccctgctgaagcagaagggcgtgaagaacagagagctgatccacatctctgactggctgccnaccctggtgaagctggccaggggccacaccaatggcaccaagcccctggatggcttcgacgtgtggaagaccatctctgagggcagcccttcccccagaattgagctgctgcacaacattgaccccaacttcgtggactctagcccntgccccaggaacagcatggcccctgccaaggatgactcctctctgccngagtactcngccttcaacacctctgtgcacgctgcnatcagacacggcaattggaagctgctgacnggctaccctggctgtggctactggttccccccnccntctcagtacaatgtgtctgagatccccagctcngacccncccaccaagaccctgtggctgtttgacattgatcgggaccctgaggagagacacgacctgtccagagagtacccccacatcgtgaccaagctgctgagcagnctgcagttctaccacaagcactcngtgcccgtgtacttccctgcccaggacccccgntgtgaccccaaggccactggggtgtggggcccctggatgtga

SEQ ID NO: 4 - DNA, 1602 bp, Homo sapiens
atgggtccgcgcggcgcggcgagcttgccccgaggccccggacctcggcggctgctcctccccgtcgtcctcccgctgctgctgctgctgttgttggcgccgccgggctcgggcgccggggccagccggccgccccacctggtcttcttgctggcagacgacctaggctggaacgacgtcggcttccacggctcccgcatccgcacgccgcacctggacgcgctggcggccggcggggtgctcctggacaactactacacgcagccgctgtgcacgccgtcgcggagccagctgctcactggccgctaccagatccgtacaggtttacagcaccaaataatctggccctgtcagcccagctgtgttcctctggatgaaaaactcctgccccagctcctaaaagaagcaggttatactacccatatggtcggaaaatggcacctgggaatgtaccggaaagaatgccttccaacccgccgaggatttgatacctactttggatatctcctgggtagtgaagattattattcccatgaacgctgtacattaattgacgctctgaatgtcacacgatgtgctcttgattttcgagatggcgaagaagttgcaacaggatataaaaatatgtattcaacaaacatattcaccaaaagggctatagccctcataactaaccatccaccagagaagcctctgtttctctaccttgctctccagtctgtgcatgagccccttcaggtccctgaggaatacttgaagccatatgactttatccaagacaagaacaggcatcactatgcaggaatggtgtcccttatggatgaagcagtaggaaatgtcactgcagctttaaaaagcagtgggctctggaacaacacggtgttcatcttttctacagataacggagggcagactttggcagggggtaataactggccccttcgaggaagaaaatggagcctgtgggaaggaggcgtccgaggggtgggctttgtggcaagccccttgctgaagcagaagggcgtgaagaaccgggagctgatccacatctctgactggctgccaacactcgtgaagctggccaggggacacaccaatggcacaaagcctctggatggcttcgacgtgtggaaaaccatcagtgaaggaagcccatcccccagaattgagctgctgcataatattgacccgaacttcgtggactcttcaccgtgtcccaggaacagcatggctggagcaaaggatgactcttctcttccagaatattcagcctttaacacatctgtccatgctgcaattagacatggaaattggaaactcctcacgggctacccaggctgtggttactggttccctccaccgtctcaatacaatgtttctgagataccctcatcagacccaccaaccaagaccctctggctctttgatattgatcgggaccctgaagaaagacatgacctgtccagagaatatcctcacatcgtcacaaagctcctgtcccgcctacagttctaccataaacactcagtccccgtgtacttccctgcacaggacccccgctgtgatcccaaggccactggggtgtggggcccttggatgtga

(19) United States
(12) Patent Application Publication
(10) Pub. No.: US 2024/0173386 A1
(43) Pub. Date: May 30, 2024

(54) ARSB VECTORS FOR TREATMENT OF MPS VI-ASSOCIATED BLINDNESS AND OTHER OCULAR MANIFESTATIONS

(71) Applicants: The University of North Carolina at Chapel Hill, Chapel Hill, NC (US); North Carolina State University, Raleigh, NC (US)

(72) Inventors: Matthew Louis Hirsch, Chapel Hill, NC (US); Liujiang Song, Chapel Hill, NC (US); Brian Christopher Gilger, Raleigh, NC (US)

(21) Appl. No.: 18/551,789

(22) PCT Filed: Mar. 22, 2022
(86) PCT No.: PCT/US2022/021249

§ 371 Date: Sep. 21, 2023

Related U.S. Application Data
(60) Provisional application No. 63/164,069, filed on Mar. 22, 2021.

(51) Int. Cl. A61K 38/46; A61K 9/00; A61K 48/00; A61P 27/02; C12N 9/16; C12N 15/86

(52) U.S. Cl. CPC A61K 38/465; A61K 9/0048; A61K 48/0033; A61P 27/02; C12N 9/16; C12N 15/86; C12Y 301/06012; C12N 2750/14122; C12N 2750/14143; C12N 2750/14152; C12N 2800/22

(57) ABSTRACT

This invention relates to vectors for delivery of ary lsulfatase B to the eye (e.g., cornea) of a subject and methods of using the same for treatment and pre-vention of corneal clouding and blindness in a subject due to mucopolysaccharidosis VI (MPS-VI) and other MPS VI-associated manifestations in the eye.

### STATEMENT OF PRIORITY

This application claims the benefit of U.S. Provisional Application Ser. No. 63/164,069, filed Mar. 22, 2021, the entire contents of which are incorporated by reference herein.

### FIELD OF THE INVENTION

This invention relates to vectors for delivery of arylsulfatase B to the eye (e.g., cornea) of a subject and methods of using the same for treatment and prevention of corneal clouding and blindness in a subject due to mucopolysaccharidosis VI (MPS VI) and other MPS VI-associated manifestations in the eye.

### BACKGROUND OF THE INVENTION

Mucopolysaccharidosis VI (MPS VI), also known as Maroteaux-Lamy syndrome, is an autosomal recessive lysosomal storage disorder caused by null or nonsense mutations in the gene encoding arylsulfatase B (ARSB), a ubiquitous intracellular, cellular surface, and secreted enzyme that catabolizes large sugar molecules known as glycosaminoglycans (GAGs). In the absence of functional ARSB, GAGs accumulate in lysosomes and disrupt the normal intracellular trafficking of lipids, sugars, and proteins causing multisystem organ damage. The incidence of MPS VI is approximately 1 in 250,000 to 600,000 and the disease is characterized by bone and joint deformities, macrocephaly, hydrocephalus, macroglossia, heart valve abnormalities, narrow airways, hepatosplenomegaly, and umbilical and inguinal hernias. An additional symptom includes clouding of the cornea which results in loss of vision. Life expectancy of individuals with MPS VI depends on the severity of symptoms. Without treatment, severely affected individuals may survive only until late childhood or adolescence. Those with milder forms of the disorder usually live until adulthood, although their life expectancy may be reduced. Heart disease and airway obstruction are major causes of death.

Current MPS VI treatments include ARSB enzyme replacement therapy (ERT) (NAGLAZYME™) via intravenous injections, which has proven useful in reducing hepatosplenomegaly, and in improving walking and stair-climbing capacity in MPS VI patients with mild disease. A more promising treatment relies on allogeneic hematopoietic stem cell transplantation (HSCT) which has been used for the past 2-3 decades in MPS VI patients. HSCT has proven successful at improving hepatosplenomegaly, airway obstruction and sleep apnea, and joint mobility and preventing further heart and lung deterioration. However, ERT and HSCT exhibit a common deficiency: which is the inability to correct MPS VI-associated maladies in privileged compartments including the joint and eye.

Regarding the ocular abnormalities, 100% of MPS VI children lose at least some vision due to corneal clouding, with severe clouding more common than in other MPS disorders. The clouding has been attributed to the abnormal presence of vacuolated corneal stromal cells. Corneal transplantation in MPS VI patients has been used to address the corneal blindness, however the high rejection rate has discouraged this treatment as standard practice.

The present invention provides vectors for expression of ARSB in the eye (e.g., cornea) and methods for treating or preventing MPS VI-associated corneal clouding and blindness and other MPS VI-associated manifestations in the eye.

### SUMMARY OF THE INVENTION

This invention is based on the finding that the use of AAV vectors for delivery of ARSB to the cornea of a subject with MPS VI is effective to express ARSB throughout the cornea. Thus, one aspect of the invention relates to a recombinant nucleic acid comprising a nucleotide sequence encoding human arylsulfatase B (ARSB), wherein the nucleotide sequence has been codon-optimized for expression in human cells.

Another aspect of the invention relates to a vector comprising a nucleotide sequence encoding ARSB, which can be codon-optimized polynucleotide of the invention. The invention includes an AAV vector genome comprising the nucleic acid of the invention, an AAV particle comprising the AAV vector genome, and a pharmaceutical composition comprising the AAV particle.

A further aspect of the invention relates to a method of producing a recombinant AAV particle comprising an AAV capsid, the method comprising: providing a cell in vitro with an AAV Cap and AAV Rep coding sequences, the AAV vector genome of the invention, and helper functions for generating a productive AAV infection; and allowing assembly of the recombinant AAV particle comprising the AAV capsid and encapsidating the AAV vector genome.

An additional aspect of the invention relates to a method of delivering ARSB to the eye (e.g., the cornea) of a subject, comprising administering to the eye of the subject an effective amount of a polynucleotide encoding ARSB, a vector that expresses ARSB, an ARSB polypeptide, and/or an AAV particle that expresses ARSB, thereby delivering ARSB to the eye of the subject.

Another aspect of the invention relates to a method of treating, slowing the progression of, or delaying the onset of MPS VI-associated corneal clouding or other ocular manifestations in a subject in need thereof, comprising administering to the eye (e.g., cornea) of the subject a therapeutically effective amount of a polynucleotide encoding ARSB, a vector that expresses ARSB, an ARSB polypeptide, and/or an AAV particle that expresses ARSB, thereby treating, slowing the progression of, or delaying the onset of MPS VI-associated corneal clouding or other ocular manifestations in the subject.

A further aspect of the invention relates to a method of delivering ARSB to a subject, comprising administering to the subject an effective amount of a polynucleotide encoding ARSB, a vector that expresses ARSB, an ARSB polypeptide, and/or an AAV particle that expresses ARSB, thereby delivering ARSB to the subject.

These and other aspects of the invention are set forth in more detail in the description of the invention below.

### BRIEF DESCRIPTION OF THE DRAWINGS

FIG. 1 shows vector maps for expressing wild-type ASRB: codon optimized ARSB (GW), and codon optimized ARSB (GS).

FIG. 2 shows a sequence alignment of wild-type ARSB, optimized sequence GS, and optimized sequence GW. Plasmid (0.5 μg) was transfected for 48 hours. For the western blot, ˜20 μg of total protein was loaded in each well. Primary antibody (ARSB Abcam (ab85727)) at 1:1000 dilution was prepared in BSA and incubated overnight at 4° C. Secondary antibody (Ab97110) at 1:5000 dilution was prepared in BSA and incubated 1 hour at room temperature. The blot was developed using chemiluminescence.

FIG. 3 shows plasmid transfections comparing two different codon optimized ARSB ORFs (GS, GW) in 293 cells by western blot.

FIG. 4 shows plasmid transfections in 293 cells (two different optimized CDNAs (GS, GW) vs WT) by ARSB activity.

FIG. 5 shows the verification of AAV8-optArsB (GS) derived ARSB activity in human corneas. 4 days post-mortem human corneas: Injection of 5×10⁹ vg AAV8-optArsB (GS); 6 day culture total harvest.

FIG. 6 shows the MPS VI feline model and study timeline.

FIG. 7 shows the MPS VI feline model study design.

FIG. 8 shows corneal images of MPS VI (homozygous or heterozygous) felines before, during, and after injection of vehicle (OD) or AAV8-optArsB (OS).

FIG. 9 shows corneal images of homozygote feline 21 days after injection of vehicle (OD) or AAV8-optArsB (OS).

FIG. 10 shows corneal images of felines before, during, and after injection of vehicle (OD) or AAV8-optArsB (OS) and then a second contralateral corneal injection of AAV8-optArsB (OD).

FIG. 11 shows ocular examination inflammatory scores before and after injections.

FIG. 12 shows corneal thickness changes during the study.

FIG. 13 shows the corneal histopathology scores after the study.

FIG. 14 shows the corneal histopathology images of the homozygote after the study.

FIG. 15 shows the corneal histopathology images of the heterozygote after the study.

FIG. 16 shows in vivo confocal microscopy of posterior stroma following AAV-optArsB in MPS VI felines.

FIG. 17 shows corneal electron microscopy following AAV-optArsB in MPS VI felines.

FIG. 18 shows alpha smooth muscle actin corneal staining following AAV-optArsB in MPS VI felines.

### DETAILED DESCRIPTION OF THE INVENTION

The present invention will now be described with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR § 1.822 and established usage. See, e.g., PatentIn User Manual, 99-102 (November 1990) (U.S. Patent and Trademark Office).

Except as otherwise indicated, standard methods known to those skilled in the art may be used for the construction of recombinant parvovirus and AAV (rAAV) constructs, packaging vectors expressing the parvovirus Rep and/or Cap sequences, and transiently and stably transfected packaging cells. Such techniques are known to those skilled in the art. See. e.g., SAMBROOK et al., MOLECULAR CLONING: A LABORATORY MANUAL 2nd Ed. (Cold Spring Harbor, NY, 1989): AUSUBEL et al., CURRENT PROTOCOLS IN MOLECULAR BIOLOGY (Green Publishing Associates, Inc. and John Wiley & Sons, Inc., New York).

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L: A, G, I or V: A or G: only L; etc. as if each such subcombination is expressly set forth herein. Moreover, such language also indicates that one or more of the specified amino acids can be disclaimed. For example, in particular embodiments the amino acid is not A, G or I: is not A: is not G or V: etc. as if each such possible disclaimer is expressly set forth herein.

### Definitions

The following terms are used in the description herein and the appended claims.

The singular forms “a” and “an” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” is to be interpreted as encompassing the recited materials or steps and those that do not materially affect the basic and novel characteristic(s) of the claimed invention (e.g., rAAV replication). Thus, the term “consisting essentially of” as used herein should not be interpreted as equivalent to “comprising.”

The term “consists essentially of” (and grammatical variants), as applied to a polynucleotide or polypeptide sequence of this invention, means a polynucleotide or polypeptide that consists of both the recited sequence (e.g., SEQ ID NO) and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together. The term “materially altered,” as applied to polynucleotides of the invention, refers to an increase or decrease in ability to express the encoded polypeptide of at least about 50% or more as compared to the expression level of a polynucleotide consisting of the recited sequence. The term “materially altered,” as applied to polypeptides of the invention, refers to an increase or decrease in enzymatic activity of at least about 50% or more as compared to the activity of a polypeptide consisting of the recited sequence.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, snake parvovirus, and B19 virus. Other autonomous parvoviruses are known to those skilled in the art. See. e.g., FIELDS et al., VIROLOGY, volume 2, chapter 69 (4th ed., Lippincott-Raven Publishers).

The genus Dependovirus contains the adeno-associated viruses (AAV), including but not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, avian AAV, bovine AAV, canine AAV, goat AAV, snake AAV, equine AAV, and ovine AAV. See. e.g., FIELDS et al., VIROLOGY, volume 2, chapter 69 (4th ed., Lippincott-Raven Publishers); and Table 1.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, snake AAV, avian AAV, bovine AAV, canine AAV, equine AAV, ovine AAV, goat AAV, shrimp AAV, and any other AAV now known or later discovered. See. e.g., FIELDS et al., VIROLOGY, volume 2, chapter 69 (4th ed., Lippincott-Raven Publishers). A number of relatively new AAV serotypes and clades have been identified (See. e.g., Gao et al., (2004) J. Virol. 78:6381: Moris et al., (2004) Virol. 33 -: 375; and Table 1).

TABLE 1

| | GenBank Accession Number |
|---|---|
| Complete Genomes | |
| Adeno-associated virus 1 | NC_002077, AF063497 |
| Adeno-associated virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, AF085716 |
| Adeno-associated virus 6 | NC_001862 |
| Avian AAV ATCC VR-865 | AY186198, AY629583, |
| | NC_004828 |
| Avian AAV strain DA-1 | NC_006263, AY629583 |
| Bovine AAV | NC_005889, AY388617 |
| Clade A | |
| AAV1 | NC_002077, AF063497 |
| AAV6 | NC_001862 |
| Hu. 48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 | AY530579 |
| (AAV9) | |
| Hu31 | AY530596 |
| Hu32 | AY530597 |
| Clonal Isolate | |
| AAV5 | Y18065, AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |

The parvovirus vectors, particles, and genomes of the present invention can be from, but are not limited to, AAV. The genomic sequences of various serotypes of AAV and the autonomous parvoviruses, as well as the sequences of the native ITRs, Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, AY631966, AX753250, EU285562, NC_001358, NC_001540, AF513851, AF513852 and AY530579; the disclosures of which are incorporated by reference herein for teaching parvovirus and AAV nucleic acid and amino acid sequences. See also. e.g., Bantel-Schaal et al., (1999) J. Virol. 73: 939; Chiorini et al., (1997) J. Virol. 71:6823: Chiorini et al., (1999) J. Virol. 73:1309; Gao et al., (2002) Proc. Nat. Acad. Sci. USA 99:11854; Moris et al., (2004) Virol. 33 -: 375-383: Mori et al., (2004) Virol. 330:375; Muramatsu et al., (1996) Virol. 221:208: Ruffing et al., (1994) J. Gen. Virol. 75:3385: Rutledge et al., (1998) J. Virol. 72:309; Schmidt et al., (2008) J. Virol. 82:8911: Shade et al., (1986) J. Virol. 58:921: Srivastava et al., (1983) J. Virol. 45:555: Xiao et al., (1999) J. Virol. 73:3994; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. An early description of the AAV1, AAV2 and AAV3 ITR sequences is provided by Xiao, X., (1996), “Characterization of Adeno-associated virus (AAV) DNA replication and integration,” Ph.D. Dissertation, University of Pittsburgh, Pittsburgh, PA (incorporated herein in its entirety).

The term “tropism” as used herein refers to entry of the virus into the cell, optionally and preferably followed by expression (e.g., transcription and, optionally, translation) of sequences carried by the viral genome in the cell, e.g., for a recombinant virus, expression of the heterologous nucleotide sequences(s). Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of AAV, gene expression from the viral genome may be from a stably integrated provirus, from a non-integrated episome, as well as any other form in which the virus may take within the cell.

As used herein, “transduction” of a cell by parvovirus or AAV refers to parvovirus/AAV-mediated transfer of genetic material into the cell. See. e.g., FIELDS et al., VIROLOGY, volume 2, chapter 69 (3d ed., Lippincott-Raven Publishers).

The terms “5′ portion” and “3′ portion” are relative terms to define a spatial relationship between two or more elements. Thus, for example, a “3′ portion” of a polynucleotide indicates a segment of the polynucleotide that is downstream of another segment. The term “3′ portion” is not intended to indicate that the segment is necessarily at the 3′ end of the polynucleotide, or even that it is necessarily in the 3′ half of the polynucleotide, although it may be. Likewise, a “5′ portion” of a polynucleotide indicates a segment of the polynucleotide that is upstream of another segment. The term “5′ portion” is not intended to indicate that the segment is necessarily at the 5′ end of the polynucleotide, or even that it is necessarily in the 5′ half of the polynucleotide, although it may be.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), and can be either single or double stranded DNA sequences.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987): the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol., 266:460 (1996): blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched: however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, an “isolated” polynucleotide (e.g., an “isolated DNA” or an “isolated RNA”) means a polynucleotide separated or substantially free from at least some of the other components of the naturally occurring organism or virus, for example, the cell or viral structural components or other polypeptides or nucleic acids commonly found associated with the polynucleotide.

Likewise, an “isolated” polypeptide means a polypeptide that is separated or substantially free from at least some of the other components of the naturally occurring organism or virus, for example, the cell or viral structural components or other polypeptides or nucleic acids commonly found associated with the polypeptide.

A “therapeutic polypeptide” is a polypeptide that may alleviate or reduce symptoms that result from an absence or defect in a protein in a cell or subject. Alternatively, a “therapeutic polypeptide” is one that otherwise confers a benefit to a subject, e.g., anti-cancer effects or improvement in transplant survivability.

As used herein, the term “modified,” as applied to a polynucleotide or polypeptide sequence, refers to a sequence that differs from a wild-type sequence due to one or more deletions, additions, substitutions, or any combination thereof.

As used herein, by “isolate” or “purify.” (or grammatical equivalents) a virus vector, it is meant that the virus vector is at least partially separated from at least some of the other components in the starting material.

By the terms “treat,” “treating,” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing,” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “heterologous nucleotide sequence” and “heterologous nucleic acid” are used interchangeably herein and refer to a sequence that is not naturally occurring in the virus. In some embodiments, the heterologous nucleic acid comprises an open reading frame that encodes a polypeptide or nontranslated RNA of interest (e.g., for delivery to a cell or subject).

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc. The region in a nucleic acid sequence or polynucleotide in which one or more regulatory elements are found may be referred to as a “regulatory region.”

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame.

The term “open reading frame (ORF),” as used herein, refers to the portion of a polynucleotide (e.g., a gene) that encodes a polypeptide, and is inclusive of the initiation start site (i.e., Kozak sequence) that initiates transcription of the polypeptide. The term “coding region” may be used interchangeably with open reading frame.

The term “codon-optimized,” as used herein, refers to a gene coding sequence that has been optimized to increase expression by substituting one or more codons normally present in a coding sequence with a codon for the same (synonymous) amino acid. In this manner, the protein encoded by the gene is identical, but the underlying nucleobase sequence of the gene or corresponding mRNA is different. In some embodiments, the optimization substitutes one or more rare codons (that is, codons for tRNA that occur relatively infrequently in cells from a particular species) with synonymous codons that occur more frequently to improve the efficiency of translation. For example, in human codon-optimization one or more codons in a coding sequence are replaced by codons that occur more frequently in human cells for the same amino acid. Codon optimization can also increase gene expression through other mechanisms that can improve efficiency of transcription and/or translation. Strategies include, without limitation, increasing total GC content (that is, the percent of guanines and cytosines in the entire coding sequence), decreasing CpG content (that is, the number of CG or GC dinucleotides in the coding sequence), removing cryptic splice donor or acceptor sites, and/or adding or removing ribosomal entry and/or initiation sites, such as Kozak sequences. Desirably, a codon-optimized gene exhibits improved protein expression, for example, the protein encoded thereby is expressed at a detectably greater level in a cell compared with the level of expression of the protein provided by the wildtype gene in an otherwise similar cell. Codon-optimization also provides the ability to distinguish a codon-optimized gene and/or corresponding mRNA from an endogenous gene and/or corresponding mRNA in vitro or in vivo.

A “vector” refers to a compound used as a vehicle to carry foreign genetic material into another cell, where it can be replicated and/or expressed. A vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene/ORF. Insertion of a vector into the target cell is referred to as transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone or a plasmid.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged.

A “TAAV vector genome” or “TAAV genome” is an AAV genome (i.e., vDNA) that comprises one or more heterologous nucleic acid sequences. rAAV vectors generally require only the 145 base ITR in cis to generate virus. All other viral sequences are dispensable and may be supplied in trans (Muzyczka (1992) Curr. Topics Microbiol. Immunol. 158:97). Typically, the rAAV vector genome will only retain the one or more ITR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV ITR sequence), optionally two ITRs (e.g., two AAV ITRs), which typically will be at the 5′ and 3′ ends of the vector genome and flank the heterologous nucleic acid, but need not be contiguous thereto. The ITRs can be the same or different from each other.

The term “terminal repeat” or “TR” includes any viral terminal repeat or synthetic sequence that forms a hairpin structure and functions as a terminal repeat (i.e., mediates the desired functions such as replication, virus packaging, integration and/or provirus rescue, and the like). The TR can be an AAV ITR or a non-AAV ITR. For example, a non-AAV ITR sequence such as those of other parvoviruses (e.g., canine parvovirus, bovine parvovirus, mouse parvovirus, porcine parvovirus, human parvovirus B-19) or the SV40 hairpin that serves as the origin of SV40 replication can be used as an ITR, which can further be modified by truncation, substitution, deletion, insertion and/or addition. Further, the ITR can be partially or completely synthetic, such as the “double-D sequence” as described in U.S. Pat. No. 5,478,745 to Samulski et al.

Parvovirus genomes have palindromic sequences at both their 5′ and 3′ ends. The palindromic nature of the sequences leads to the formation of a hairpin structure that is stabilized by the formation of hydrogen bonds between the complementary base pairs. This hairpin structure is believed to adopt a “Y” or a “T” shape. See, e.g., FIELDS et al., VIROLOGY, volume 2, chapters 69 & 70 (4th ed., Lippincott-Raven Publishers).

An “AAV inverted terminal repeat” or “AAV ITR” may be from any AAV, including but not limited to serotypes 1, 2, 3a, 3b, 4, 5, 6, 7, 8, 9, 10, 11, or 13, snake AAV, avian AAV, bovine AAV, canine AAV, equine AAV, ovine AAV, goat AAV, shrimp AAV, or any other AAV now known or later discovered (see. e.g., Table 1). An AAV ITR need not have the native terminal repeat sequence (e.g., a native AAV ITR sequence may be altered by insertion, deletion, truncation and/or missense mutations), as long as the terminal repeat mediates the desired functions, e.g., replication, virus packaging, persistence, and/or provirus rescue, and the like.

The virus vectors of the invention can further be “targeted” virus vectors (e.g., having a directed tropism) and/or a “hybrid” parvovirus (i.e., in which the viral ITRs and viral capsid are from different parvoviruses) as described in international patent publication WO 00/28004 and Chao et al., (2000) Mol. Therapy 2:619.

Further, the viral capsid or genomic elements can contain other modifications, including insertions, deletions and/or substitutions.

As used herein, the term “amino acid” encompasses any naturally occurring amino acids, modified forms thereof, and synthetic amino acids.

Naturally occurring, levorotatory (L-) amino acids are shown in Table 2.

TABLE 2

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 3) or can be an amino acid that is modified by post-translation modification (e.g., acetylation, amidation, formylation, hydroxylation, methylation, phosphorylation or sulfatation).

TABLE 3
Amino Acid Residue Derivatives

| Modified Amino Acid Residue | Abbreviation |
|---|---|
| 2-Aminoadipic acid | Aad |
| 3-Aminoadipic acid | bAad |
| beta-Alanine, beta-Aminoproprionic | bAla |
| acid | Abu |
| 2-Aminobutyric acid | 4Abu |
| 4-Aminobutyric acid, Piperidinic acid | Acp |
| 6-Aminocaproic acid | Ahe |
| 2-Aminoheptanoic acid | Aib |
| 2-Aminoisobutyric acid | bAib |
| 3-Aminoisobutyric acid | Apm |
| 2-Aminopimelic acid | t-BuA |
| t-butylalanine | Cit |
| Citrulline | Cha |
| Cyclohexylalanine | Dbu |
| 2,4-Diaminobutyric acid | Des |
| Desmosine | Dpm |
| 2,2′-Diaminopimelic acid | Dpr |
| 2,3-Diaminoproprionic acid | EtGly |
| N-Ethylglycine | EtAsn |
| N-Ethylasparagine | hArg |
| Homoarginine | hCys |
| Homocysteine | hSer |
| Homoserine | Hyl |
| Hydroxylysine | aHyl |
| Allo-Hydroxylysine | 3Hyp |
| 3-Hydroxyproline | 4Hyp |
| 4-Hydroxyproline | Ide |
| Isodesmosine | aIle |
| allo-Isoleucine | MSO |
| Methionine sulfoxide | MeGly |
| N-Methylglycine, sarcosine | MeIle |
| N-Methylisoleucine | MeLys |
| 6-N-Methyllysine | MeVal |
| N-Methylvaline | 2-Nal |
| 2-Naphthylalanine | Nva |
| Norvaline | Nle |
| Norleucine | Orn |
| Ornithine | Phe(4-Cl) |
| 4-Chlorophenylalanine | Phe(2-F) |
| 2-Fluorophenylalanine | Phe(3-F) |
| 3-Fluorophenylalanine | Phe(4-F) |
| 4-Fluorophenylalanine | Phg |
| Phenylglycine | Thi |
| Beta-2-thienylalanine | |

Further, the non-naturally occurring amino acid can be an “unnatural” amino acid as described by Wang et al., (2006) Annu. Rev. Biophys. Biomol. Struct. 35:225-49. These unnatural amino acids can advantageously be used to chemically link molecules of interest to the AAV capsid protein.

The term “template” or “substrate” is used herein to refer to a polynucleotide sequence that may be replicated to produce the parvovirus viral DNA. For the purpose of vector production, the template will typically be embedded within a larger nucleotide sequence or construct, including but not limited to a plasmid, naked DNA vector, bacterial artificial chromosome (BAC), yeast artificial chromosome (YAC) or a viral vector (e.g., adenovirus, herpesvirus, Epstein-Barr Virus, AAV, baculoviral, retroviral vectors, and the like). Alternatively, the template may be stably incorporated into the chromosome of a packaging cell.

As used herein, parvovirus or AAV “Rep coding sequences” indicate the nucleic acid sequences that encode the parvoviral or AAV non-structural proteins that mediate viral replication and the production of new virus particles. The parvovirus and AAV replication genes and proteins have been described in. e.g., FIELDS et al., VIROLOGY, volume 2, chapters 69 & 70 (4th ed., Lippincott-Raven Publishers).

The “Rep coding sequences” need not encode all of the parvoviral or AAV Rep proteins. For example, with respect to AAV, the Rep coding sequences do not need to encode all four AAV Rep proteins (Rep78, Rep 68, Rep52 and Rep40), in fact, it is believed that AAV5 only expresses the spliced Rep68 and Rep40 proteins. In representative embodiments, the Rep coding sequences encode at least those replication proteins that are necessary for viral genome replication and packaging into new virions. The Rep coding sequences will generally encode at least one large Rep protein (i.e., Rep78/68) and one small Rep protein (i.e., Rep52/40). In particular embodiments, the Rep coding sequences encode the AAV Rep78 protein and the AAV Rep52 and/or Rep40 proteins. In other embodiments, the Rep coding sequences encode the Rep68 and the Rep52 and/or Rep40 proteins. In a still further embodiment, the Rep coding sequences encode the Rep68 and Rep52 proteins, Rep68 and Rep40) proteins, Rep78 and Rep52 proteins, or Rep78 and Rep40) proteins.

As used herein, the term “large Rep protein” refers to Rep68 and/or Rep78. Large Rep proteins of the claimed invention may be either wild-type or synthetic. A wild-type large Rep protein may be from any parvovirus or AAV, including but not limited to serotypes 1, 2, 3a, 3b, 4, 5, 6, 7, 8, 9, 10, 11, or 13, or any other AAV now known or later discovered (see. e.g., Table 1). A synthetic large Rep protein may be altered by insertion, deletion, truncation and/or missense mutations.

Those skilled in the art will further appreciate that it is not necessary that the replication proteins be encoded by the same polynucleotide. For example, for MVM, the NS-1 and NS-2 proteins (which are splice variants) may be expressed independently of one another. Likewise, for AAV, the p19 promoter may be inactivated and the large Rep protein(s) expressed from one polynucleotide and the small Rep protein(s) expressed from a different polynucleotide. Typically, however, it will be more convenient to express the replication proteins from a single construct. In some systems, the viral promoters (e.g., AAV p19 promoter) may not be recognized by the cell, and it is therefore necessary to express the large and small Rep proteins from separate expression cassettes. In other instances, it may be desirable to express the large Rep and small Rep proteins separately, i.e., under the control of separate transcriptional and/or translational control elements. For example, it may be desirable to control expression of the large Rep proteins, so as to decrease the ratio of large to small Rep proteins. In the case of insect cells, it may be advantageous to down-regulate expression of the large Rep proteins (e.g., Rep78/68) to avoid toxicity to the cells (see. e.g., Urabe et al., (2002) Human Gene Therapy 13:1935).

As used herein, the parvovirus or AAV “cap coding sequences” encode the structural proteins that form a functional parvovirus or AAV capsid (i.e., can package DNA and infect target cells). Typically, the cap coding sequences will encode all of the parvovirus or AAV capsid subunits, but less than all of the capsid subunits may be encoded as long as a functional capsid is produced. Typically, but not necessarily, the cap coding sequences will be present on a single nucleic acid molecule.

The capsid structure of autonomous parvoviruses and AAV are described in more detail in BERNARD N. FIELDS et al., VIROLOGY, volume 2, chapters 69 & 70 (4th ed., Lippincott-Raven Publishers).

### Vectors Expressing ARSB

The present invention provides vectors, e.g., parvovirus vectors, e.g., AAV vectors, that comprise a nucleotide sequence encoding ARSB and are capable of expressing ARSB in the cornea of a subject.

One aspect of the invention relates to a recombinant nucleic acid comprising, consisting essentially of, or consisting of a nucleotide sequence encoding human arylsulfatase B (ARSB), wherein the nucleotide sequence has been codon-optimized for expression in human cells. In certain embodiments, the nucleic acid is a non-naturally occurring sequence. In some embodiments, the nucleic acid comprises, consists essentially of, or consists of a nucleotide sequence that is at least 90% identical to SEQ ID NO:1 or SEQ ID NO:2, e.g., at least 91%, 92%, 93%, 94%, 95%, 96%, 97%, 98%, or 99% identical to SEQ ID NO:1 or SEQ ID NO:2. In some embodiments, the nucleic acid comprises, consists essentially of, or consists of the nucleotide sequence of SEQ ID NO: 1 or SEQ ID NO:2. In some embodiments, the nucleic acid comprises at least 10 contiguous nucleotides of SEQ ID NO:1 or SEQ ID NO:2, e.g., at least 10, 25, 50, 100, 200, 300, 400, 500, 600, 700, 800, 900, 1000, 1100, 1200, 1300, 1400, 1500, 1600, 1700, 1800 or more.

(GS)
SEQ ID NO: 1
ATGGGGCCAAGAGGAGCTGCATCATTACCCAGGGGACCAGGGCCCAGGAGACTGCTGCTGCCTGTG
GTGCTGCCCCTGTTATTACTGCTGCTGCTGGCTCCTCCTGGCAGCGGAGCTGGAGCCTCCCGCCCTCCT
CACCTGGTGTTCCTGCTGGCCGACGATCTGGGCTGGAACGACGTGGGCTTCCACGGCAGCAGGATCA
GAACCCCTCACCTGGATGCCCTGGCTGCTGGAGGCGTGCTGCTGGACAATTACTACACCCAGCCTCTG
TGCACCCCCTCTAGGAGCCAGCTGCTGACCGGCAGGTACCAGATCAGAACCGGCCTGCAGCACCAGA
TCATCTGGCCTTGCCAGCCTTCCTGCGTGCCTCTGGACGAGAAGCTGCTGCCTCAGCTGCTGAAGGAG
GCTGGCTACACCACCCACATGGTGGGCAAGTGGCACCTGGGCATGTACAGGAAGGAGTGTCTGCCTA
CCCGGAGGGGATTCGATACCTACTTCGGCTACCTGCTGGGCTCTGAGGACTACTACAGCCACGAGAGG
TGCACCCTGATCGATGCCCTGAACGTGACCAGATGTGCCCTGGATTTCCGGGACGGAGAGGAGGTGG
CTACCGGATACAAGAACATGTACTCCACCAATATCTTCACCAAGAGAGCCATCGCCCTGATCACCAATCA
CCCCCCTGAGAAGCCTCTGTTCCTGTACCTGGCCCTGCAGTCTGTGCACGAGCCTCTGCAGGTGCCCG
AGGAGTACCTGAAGCCCTACGATTTCATCCAGGACAAGAACCGGCACCACTACGCCGGCATGGTGAG
CCTGATGGACGAGGCTGTGGGAAATGTGACCGCCGCCCTGAAGAGCTCCGGACTGTGGAACAATACC
GTGTTCATCTTCTCCACCGATAACGGAGGACAGACCCTGGCTGGAGGAAACAATTGGCCTCTGAGGG
GAAGGAAGTGGTCCCTGTGGGAGGGAGGCGTGAGAGGAGTGGGATTCGTGGCTTCTCCTCTGCTGA
AGCAGAAGGGCGTGAAGAACAGAGAGCTGATCCACATCAGCGACTGGCTGCCTACCCTGGTGAAGC
TGGCCAGGGGCCACACCAATGGCACCAAGCCCCTGGATGGCTTCGACGTGTGGAAGACCATCTCTGA
GGGCTCCCCTTCTCCCCGCATCGAGCTGCTGCACAACATCGATCCCAATTTCGTGGACTCTAGCCCTTG
CCCCAGGAACAGCATGGCCCCTGCCAAGGACGATTCCTCTCTGCCCGAGTACTCCGCCTTCAACACCT
CTGTGCACGCTGCTATCAGGCACGGCAATTGGAAGCTGCTGACCGGCTACCCTGGCTGTGGCTACTGG
TTCCCCCCTCCCAGCCAGTACAACGTGAGCGAGATCCCCAGCTCCGACCCTCCCACCAAGACCCTGTG
GCTGTTCGACATCGATCGGGACCCTGAGGAGCGCCACGATCTGTCCAGGGAGTACCCCCACATCGTGA
CCAAGCTGCTGAGCAGGCTGCAGTTCTACCACAAGCACAGCGTGCCCGTGTACTTCCCCGCCCAGGA
CCCCCGGTGCGACCCCAAGGCCACCGGCGTGTGGGGCCCCTGGATGTGA
(GW)
SEQ ID NO: 2
ATGGGGCCTAGAGGGGCTGCTAGCCTGCCTAGAGGCCCTGGACCTAGAAGGCTGCTGCTGCCTGTG
GTGCTTCCTCTCCTGCTGCTTCTTTTACTGGCCCCCCCTGGCTCTGGGGCTGGGGCTAGCAGACCCCC
CCATTTGGTGTTCCTGCTGGCTGATGACCTGGGCTGGAATGATGTGGGCTTTCATGGCAGCAGAATC
AGAACCCCCCATCTTGATGCCCTGGCTGCTGGGGGGGTGCTGCTGGACAACTACTACACACAGCCCC
TGTGCACCCCTAGCAGAAGCCAACTcCTGACTGGCAGATATCAGATCAGAACTGGCCTGCAGCATCA
GATCATCTGGCCCTGTCAGCCTAGCTGTGTGCCCCTGGATGAGAAGCTGCTGCCTCAGCTGCTGAAG
GAGGCTGGCTACACCACCCACATGGTGGGCAAGTGGCACCTGGGCATGTACAGAAAGGAGTGCCT
GCCCACAAGAAGAGGCTTTGACACCTACTTTGGCTACCTGCTGGGCTCTGAGGACTACTACAGCCAT
GAGAGATGCACCCTGATTGATGCCCTGAATGTGACAAGATGTGCCCTGGACTTCAGAGATGGGGAA
GAGGTGGCCACTGGCTACAAGAACATGTACAGCACCAACATCTTCACCAAGAGAGCCATTGCCCTG
ATCACCAACCATCCCCCTGAAAAGCCCCTGTTCCTGTACCTGGCCCTGCAGTCTGTGCATGAGCCCCT
GCAAGTGCCTGAGGAGTACCTGAAGCCCTATGACTTCATCCAAGACAAGAACAGACACCACTATGCT
GGCATGGTGAGCCTGATGGATGAGGCTGTGGGCAATGTGACAGCTGCCCTGAAGAGCTCTGGCCT
GTGGAACAACACAGTGTTCATCTTCAGCACAGACAATGGGGGCCAAACCCTGGCTGGGGGCAATAA
TTGGCCCCTGAGAGGCAGAAAGTGGAGCCTGTGGGAGGGGGGGGTGAGAGGGGTGGGCTTTGTG
GCTAGCCCCCTGCTGAAGCAGAAGGGGGTGAAGAACAGAGAGCTGATCCACATCTCTGACTGGCTG
CCCACCCTGGTGAAGCTGGCTAGAGGCCACACCAATGGCACCAAGCCCCTGGATGGCTTTGATGTG
TGGAAGACCATCTCTGAGGGCAGCCCTAGCCCTAGAATTGAGCTGCTGCACAACATTGACCCCAACT
TTGTGGACAGCAGCCCCTGCCCTAGAAACAGCATGGCCCCTGCCAAGGATGACAGCAGCCTGCCTG
AGTACTCTGCCTTCAACACCTCTGTGCACGCTGCCATCAGACACGGCAACTGGAAGCTGCTGACTGG
CTACCCTGGCTGTGGCTACTGGTTCCCCCCCCCTTCTCAGTACAATGTGTCTGAGATCCCTAGCTCTG
ACCCCCCCACCAAAACCCTGTGGCTGTTTGACATTGACAGAGACCCTGAGGAGAGACAcGACCTGAG
CAGAGAGTACCCCCACATTGTGACCAAGCTGCTGAGCAGACTGCAGTTCTACCACAAGCACTCTGTG
CCTGTGTACTTCCCTGCCCAAGACCCTAGATGTGACCCCAAGGCCACTGGGGTGTGGGGCCCCTGGA
TGTGA

Methods of codon optimizing a nucleotide sequence to maximize expression in an organism are well known in the art and can be carried out using software available to the public. The wild-type sequence of human ARSB is known in the art and can be found in databases such as GenBank. Examples of human ARSB accession numbers include NM_000046.5 and NM_198709.3, incorporated by reference herein in their entirety.

The invention also provides a vector, e.g., viral vector genome, comprising the ARSB nucleic acid of the invention. In certain embodiments, the ARSB nucleic acid is the wild-type human ARSB sequence or a codon-optimized sequence. The viral vector genome may be a parvovirus vector genome, e.g., an AAV vector genome. In some embodiments, the AAV vector genome is an AAV2, AAV8, or AAV9 vector genome.

The vector may further comprise a promoter operably linked to the ARSB nucleic acid. In some embodiments, the promoter may be a constitutive promoter, e.g., a CMV promoter or EF-1α promoter. In other embodiments, the promoter may be a tissue-specific or preferred promoter. The invention further provides a cell in vitro comprising the vector, e.g., AAV vector genome, of the invention, e.g., stably incorporated into the genome of the cell. The invention further provides a viral particle, e.g., a recombinant parvovirus particle (e.g., a recombinant AAV particle) comprising the viral vector genome of the invention. Viral vectors and viral particles are discussed further below.

In certain embodiments, the viral vector exhibits a modified tropism due to the presence of the capsid protein of the invention. In one embodiment, the parvovirus vector exhibits systemic tropism for the cornea. In other embodiments, the parvovirus vector has reduced tropism for liver compared to a virus vector comprising a wild-type capsid protein.

### Methods of Producing Virus Vectors

The present invention further provides methods of producing virus vectors. In one particular embodiment, the present invention provides a method of producing a recombinant parvovirus particle, comprising providing to a cell permissive for parvovirus replication: (a) a recombinant parvovirus template comprising (i) a nucleic acid encoding ARSB, and (ii) a parvovirus ITR: (b) a polynucleotide comprising Rep and Cap coding sequences: under conditions sufficient for the replication and packaging of the recombinant parvovirus template: whereby recombinant parvovirus particles are produced in the cell. Conditions sufficient for the replication and packaging of the recombinant parvovirus template can be, e.g., the presence of AAV sequences sufficient for replication of the parvovirus template and encapsidation into parvovirus capsids (e.g., parvovirus rep sequences and parvovirus cap sequences) and helper sequences from adenovirus and/or herpesvirus. In particular embodiments, the parvovirus template comprises two parvovirus ITR sequences, which are located 5′ and 3′ to the heterologous nucleic acid sequence, although they need not be directly contiguous thereto.

In some embodiments, the recombinant parvovirus template comprises an ITR that is not resolved by Rep to make duplexed AAV vectors as described in international patent publication WO 01/92551.

The parvovirus template and parvovirus rep and cap sequences are provided under conditions such that virus vector comprising the parvovirus template packaged within the parvovirus capsid is produced in the cell. The method can further comprise the step of collecting the virus vector from the cell. The virus vector can be collected from the medium and/or by lysing the cells.

The cell can be a cell that is permissive for parvoviral viral replication. Any suitable cell known in the art may be employed. In particular embodiments, the cell is a mammalian cell (e.g., a primate or human cell). As another option, the cell can be a trans-complementing packaging cell line that provide functions deleted from a replication-defective helper virus, e.g., 293 cells or other E1a trans-complementing cells.

The parvovirus replication and capsid sequences may be provided by any method known in the art. Current protocols typically express the parvovirus rep/cap genes on a single plasmid. The parvovirus replication and packaging sequences need not be provided together, although it may be convenient to do so. The parvovirus rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). EBV vectors may also be employed to express the parvovirus cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the replcap sequences may be stably incorporated into a cell.

Typically the parvovirus rep/cap sequences will not be flanked by the TRs, to prevent rescue and/or packaging of these sequences.

The parvovirus template can be provided to the cell using any method known in the art. For example, the template can be supplied by a non-viral (e.g., plasmid) or viral vector. In particular embodiments, the parvovirus template is supplied by a herpesvirus or adenovirus vector (e.g., inserted into the E1a or E3 regions of a deleted adenovirus). As another illustration, Palombo et al., (1998) J. Virology 72:5025, describes a baculovirus vector carrying a reporter gene flanked by the AAV TRs. EBV vectors may also be employed to deliver the template, as described above with respect to the rep/cap genes.

In another representative embodiment, the parvovirus template is provided by a replicating rAAV virus. In still other embodiments, an AAV provirus comprising the parvovirus template is stably integrated into the chromosome of the cell.

To enhance virus titers, helper virus functions (e.g., adenovirus or herpesvirus) that promote a productive parvovirus infection can be provided to the cell. Helper virus sequences necessary for parvovirus replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient parvovirus production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by ITRs.

Those skilled in the art will appreciate that it may be advantageous to provide the parvovirus replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the parvovirus rep/cap sequences and the adenovirus helper sequences are supplied by a single adenovirus helper vector. This vector can further comprise the parvovirus template. The parvovirus rep/cap sequences and/or the parvovirus template can be inserted into a deleted region (e.g., the E1a or E3 regions) of the adenovirus.

In a further embodiment, the parvovirus rep/cap sequences and the adenovirus helper sequences are supplied by a single adenovirus helper vector. According to this embodiment, the parvovirus template can be provided as a plasmid template.

In another illustrative embodiment, the parvovirus replcap sequences and adenovirus helper sequences are provided by a single adenovirus helper vector, and the parvovirus template is integrated into the cell as a provirus. Alternatively, the parvovirus template is provided by an EBV vector that is maintained within the cell as an extrachromosomal element (e.g., as an EBV based nuclear episome).

In a further exemplary embodiment, the parvovirus rep/cap sequences and adenovirus helper sequences are provided by a single adenovirus helper. The parvovirus template can be provided as a separate replicating viral vector. For example, the parvovirus template can be provided by a parvovirus particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The parvovirus replcap sequences and, if present, the AAV template are embedded in the adenovirus backbone and are flanked by the 5′ and 3′ cis sequences, so that these sequences may be packaged into adenovirus capsids. As described above, the adenovirus helper sequences and the parvovirus replcap sequences are generally not flanked by ITRs so that these sequences are not packaged into the parvovirus virions.

Zhang et al., ((2001) Gene Ther. 18:704-12) describe a chimeric helper comprising both adenovirus and the AAV rep and cap genes.

Herpesvirus may also be used as a helper virus in parvovirus packaging methods. Hybrid herpesviruses encoding the parvovirus Rep protein(s) may advantageously facilitate scalable parvovirus vector production schemes. A hybrid herpes simplex virus type I (HSV-1) vector expressing the AAV-2 rep and cap genes has been described (Conway et al., (1999) Gene Ther. 6:986 and WO 00/17377.

As a further alternative, the virus vectors of the invention can be produced in insect cells using baculovirus vectors to deliver the rep/cap genes and parvovirus template as described, for example, by Urabe et al., (2002) Human Gene Ther. 13:1935-43.

Parvovirus vector stocks free of contaminating helper virus may be obtained by any method known in the art. For example, parvovirus and helper virus may be readily differentiated based on size. Parvovirus may also be separated away from helper virus based on affinity for a heparin substrate (Zolotukhin et al., (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of parvovirus. Adenovirus mutants defective for late gene expression are known in the art (e.g., ts100K and ts149 adenovirus mutants).

### Vectors

The vectors of the present invention, e.g., viral vectors or non-viral vectors, are useful for the delivery of nucleic acids to cells in vitro, ex vivo, and in vivo. In particular, the vectors can be advantageously employed to deliver or transfer nucleic acids to animal, including mammalian, cells. In particular, the vectors of the present invention are useful for the delivery of a nucleic acid encoding ARSB to the cornea or other parts of the eye of a subject.

Cornea targeted AAV gene therapy has been investigated in animal models primarily following two routes of administration: topical applications in wound healing assay's and direct injection to the corneal stroma. Regarding topical applications, AAV serotype 9 (AAV9) was reported most efficient for stromal transduction, however, this was nearly entirely localized to the epithelial/stromal boundary (Sharma et al., Exp. Eye Res. 91(3):440 (2010)). Regarding AAV gene delivery following intrastromal injection into human cornea explants, it was observed that AAV8 was more efficient than AAV2 or AAVI for stromal transduction, which encompassed multiple cell types including CD34⁺ keratocytes and macrophages (Hippert et al., PLOS One. 7(4):e35318 (2012)). Importantly, both of these routes of drug administration observed no deleterious consequences related to the AAV vector (Sharma et al., Exp. Eye Res. 91(3):440 (2010): Hippert et al., PLoS One. 7(4): e35318 (2012): Mohan et al., PLoS One 6(10): e26432 (2011)).

It will be understood by those skilled in the art that the nucleic acid encoding ARSB can be operably associated with appropriate control sequences. For example, the nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the ARSB nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the nucleic acid sequence. Inducible promoters/enhancer elements for gene delivery can be tissue-specific or -preferred promoter/enhancer elements, and include eye specific or preferred (including retina-specific and cornea-specific) promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the nucleic acid sequence is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The vectors of the invention can be viral vectors or non-viral vectors, e.g., parvovirus vectors, e.g., AAV vectors. The AAV vectors may be any AAV serotype. In some embodiments, the AAV vector is an AAV2, AAV8, or AAV9 vector. In some embodiments, the AAV vector is a hybrid vector, e.g., one having a capsid protein from one serotype and a genome from another serotype or one having a synthetic capsid protein. In certain embodiments, the vector comprises a hybrid capsid with an altered tropism. In one example the hybrid capsid comprising a glycan binding site (e.g., a galactose binding site) from one serotype (e.g., AAV9) in a capsid sequence from another serotype (e.g., AAV8) (see, e.g., WO 2014/144229, incorporated by reference herein in its entirety).

The vectors according to the present invention provide a means for delivering ARSB nucleic acids into a broad range of cells, including dividing and non-dividing cells. The vectors can be employed to deliver the nucleic acid to a cell in vitro, e.g., to produce a polypeptide in vitro or for ex vivo gene therapy. The vectors are additionally useful in a method of delivering the nucleic acid to a subject in need thereof, e.g., to express ARSB. In this manner, the polypeptide can be produced in vivo in the subject. The subject can be in need of the polypeptide because the subject has a deficiency of the polypeptide. Further, the method can be practiced because the production of the polypeptide in the subject may impart some beneficial effect.

The vectors can also be used to produce ARSB in cultured cells or in a subject (e.g., using the subject as a bioreactor to produce the polypeptide or to observe the effects of the polypeptide on the subject, for example, in connection with screening methods).

The vectors of the present invention can be employed to deliver a nucleic acid encoding ARSB to treat and/or prevent any disease state for which it is beneficial to deliver ARSB, e.g., MPS VI.

Vectors according to the instant invention find use in diagnostic and screening methods, whereby the ARSB nucleic acid is transiently or stably expressed in a cell culture system, in an organ or organ culture (e.g., an eye), or alternatively, a transgenic animal model.

The vectors of the present invention can also be used for various non-therapeutic purposes, including but not limited to use in protocols to assess gene targeting, clearance, transcription, translation, etc., as would be apparent to one skilled in the art. The vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

Alternatively, the vector may be administered to a cell ex vivo, e.g., a corneal explant, a corneal cell, or a limbal stem cell, and the altered cell or explant is administered to the subject. The vector comprising the ARSB nucleic acid is introduced into the cell, and the cell is administered to the subject, where the nucleic acid can be expressed.

### Subjects, Pharmaceutical Formulations, and Modes of Administration

Vectors, e.g., virus vectors and capsids according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles and adults.

In particular embodiments, the present invention provides a pharmaceutical composition comprising a vector of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The vector may be introduced into the cells, e.g., at the appropriate multiplicity of infection for a viral vector, according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, more preferably at least about 10⁵ infectious units are introduced to the cell.

The cell(s) into which the vector is introduced can be of any type, including but not limited to cells of the eye (including retinal cells, retinal pigment epithelium, and corneal cells (e.g., keratocytes, limbal stem cells, epithelial cells, and endothelial cells). Moreover, the cell can be from any species of origin, as indicated above.

The vector can be introduced into cells in vitro for the purpose of administering the modified cell to a subject. In particular embodiments, the cells have been removed from a subject, the vector is introduced therein, and the cells are then administered back into the subject. Methods of removing cells from subject for manipulation ex vivo, followed by introduction back into the subject are known in the art (see. e.g., U.S. Pat. No. 5,399,346). Alternatively, the vector can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the vector are administered to the subject in a treatment effective or prevention effective amount in combination with a pharmaceutical carrier.

A further aspect of the invention is a method of administering the vector to subjects. Administration of the virus vectors according to the present invention to a human subject or an animal in need thereof can be by any means known in the art. Optionally, the vector is delivered in a treatment effective or prevention effective dose in a pharmaceutically acceptable carrier.

Dosages of the vector to be administered to a subject depend upon the mode of administration, the disease or condition to be treated and/or prevented, the individual subject's condition, the particular vector, and the nucleic acid to be delivered, and the like, and can be determined in a routine manner. Exemplary doses for achieving therapeutic effects for viral vectors are titers of at least about 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸ transducing units, optionally about 10⁸ to about 10¹⁵ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration to the eye include intrastromal, topical, intracameral, intravitreal, subconjunctival, suprachoroidal, subtenon, retrobulbar, and subretinal.

In some embodiments, the vector may be delivered systemically or locally e.g., intracameral administration for delivery to the anterior chamber of the eye or intranasal administration for delivery to the brain.

Delivery to a target tissue can also be achieved by delivering a depot comprising the vector. In representative embodiments, a depot comprising the vector is implanted into the cornea or other tissue of the eye or the tissue can be contacted with a film or other matrix comprising the vector. Such implantable matrices or substrates are described in U.S. Pat. No. 7,201,898.

In particular embodiments, a vector according to the present invention is administered to the cornea to treat, slow the progression of, delay the onset of, and/or prevent corneal clouding and/or blindness associated with MPS VI. In other embodiments, the vector is administered to the cornea and/or other parts of the eye to treat other ocular manifestations of MPS VI, including without limitation glaucoma, optic nerve compression or degeneration, retinal degeneration, dilation issues, and poor muscle control. In some embodiments, corneal administration may result in delivery of ARSB to other portions of the eye. In other embodiments, the vector may be delivered to other parts of the eye, e.g., by topical, intracameral, intravitreal, subconjunctival, suprachoroidal, or subretinal administration.

Thus, as one aspect, the invention further encompasses a method of delivering ARSB to the cornea of a subject, comprising administering to the cornea of the subject an effective amount of a vector, e.g., an AAV particle, that expresses ARSB, thereby delivering ARSB to the cornea of the subject.

In an additional aspect, the invention further encompasses a method of delivering ARSB to the eye of a subject, comprising administering to the eye of the subject an effective amount of a vector, e.g., an AAV particle, that expresses ARSB, thereby delivering ARSB to the eye of the subject.

In another aspect, the invention further encompasses a method of treating, slowing the progression of, delaying the onset of, and/or preventing MPS VI-associated corneal clouding in a subject in need thereof, comprising administering to the cornea of the subject a therapeutically effective amount of a vector, e.g., an AAV particle that expresses ARSB, thereby treating, slowing the progression of, delaying the onset of, and/or preventing MPS VI-associated corneal clouding in the subject.

In another aspect, the invention further encompasses a method of treating, slowing the progression of, delaying the onset of, and/or preventing MPS VI-associated corneal clouding or other ocular manifestations of MPS VI in a subject in need thereof, comprising administering to the eye of the subject a therapeutically effective amount of a vector, e.g., an AAV particle that expresses ARSB, thereby treating, slowing the progression of, delaying the onset of, and/or preventing MPS VI-associated corneal clouding or other ocular manifestations of MPS VI in the subject.

In a further aspect, the invention further encompasses a method of delivering ARSB to a cornea in vitro or ex vivo, e.g., prior to transplantation in a subject in need thereof, comprising contacting the cornea with an effective amount of a vector, e.g., an AAV particle that expresses ARSB, thereby delivering ARSB to the cornea. In some embodiments, the cornea may be incubated with the vector or the vector may be injected into the cornea.

In another aspect, the invention encompasses a method of delivering ARSB to a subject, comprising administering to the subject an effective amount of a vector, e.g., an AAV particle that expresses ARSB, thereby delivering ARSB to the subject.

It will be understood that the methods of the invention, although described using a vector to express ASRB, may also be carried by other means of delivering or expressing ASRB. For example, a polynucleotide, e.g., a DNA or RNA molecule, encoding ASRB may be delivered to the eye in the absence of a vector. The polynucleotide may be part of a complex that enhances delivery and/or stability of the polynucleotide, e.g., a polynucleotide/lipid complex or polynucleotide/protein complex. In another example, an ARSB polypeptide may be delivered to the eye. The ARSB polypeptide may be a wild-type polypeptide or a modified polypeptide, e.g., for enhanced delivery and/or stability of the polypeptide. The ARSB polypeptide may be part of a complex that enhances delivery and/or stability of the polypeptide.

In the methods of the invention, the subject may be one has been diagnosed with MPS VI or is suspected of having MPS VI. In certain embodiments, the subject is an infant or child, e.g., less than 18 years old, e.g., less than 18, 17, 16, 15, 14, 13, 12, 11, 10, 9, 8, 7, 6, or 5 years old. In certain embodiments, the subject is an adult, e.g., at least 18 years old. In some embodiments, the subject has not developed clouding of the cornea. In other embodiments, the subject has at least partial clouding of the cornea, e.g., the subject's eyesight has been reduced by less than about 10%, e.g., less than about 10%, 20%, 30%, 40%, 50%, 60%, 70%, 80%, or 90% relative to a subject that does not have MPS VI.

Injectables can be prepared in conventional forms, either as liquid solutions or suspensions, solid forms suitable for solution or suspension in liquid prior to injection, or as emulsions. Alternatively, one may administer the vector, e.g., virus vector and/or virus capsids of the invention in a local manner, for example, in a depot or sustained-release formulation. Further, the virus vector and/or virus capsid can be delivered adhered to a surgically implantable matrix (e.g., as described in U.S. Patent Publication No. 2004-0013645).

Having described the present invention, the same will be explained in greater detail in the following examples, which are included herein for illustration purposes only, and which are not intended to be limiting to the invention.

### EXAMPLE 1

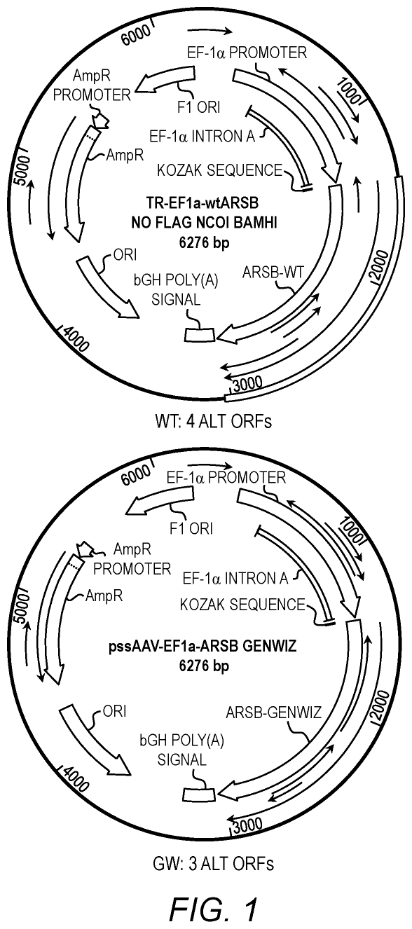

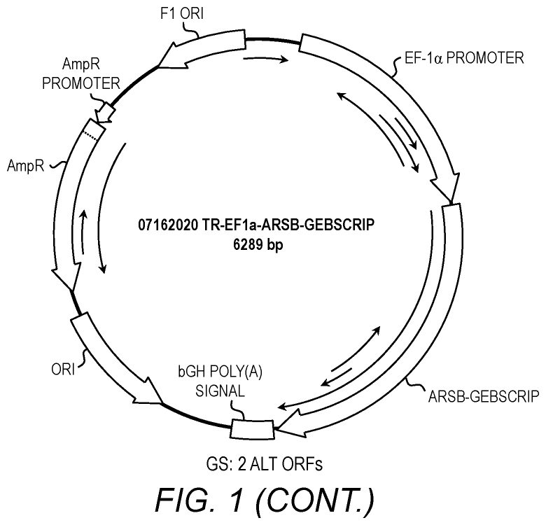

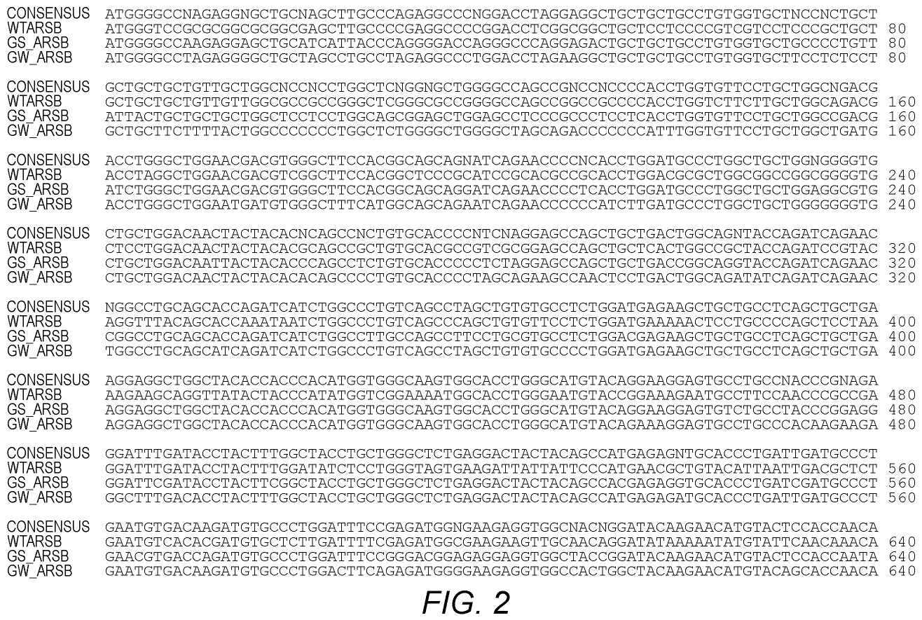

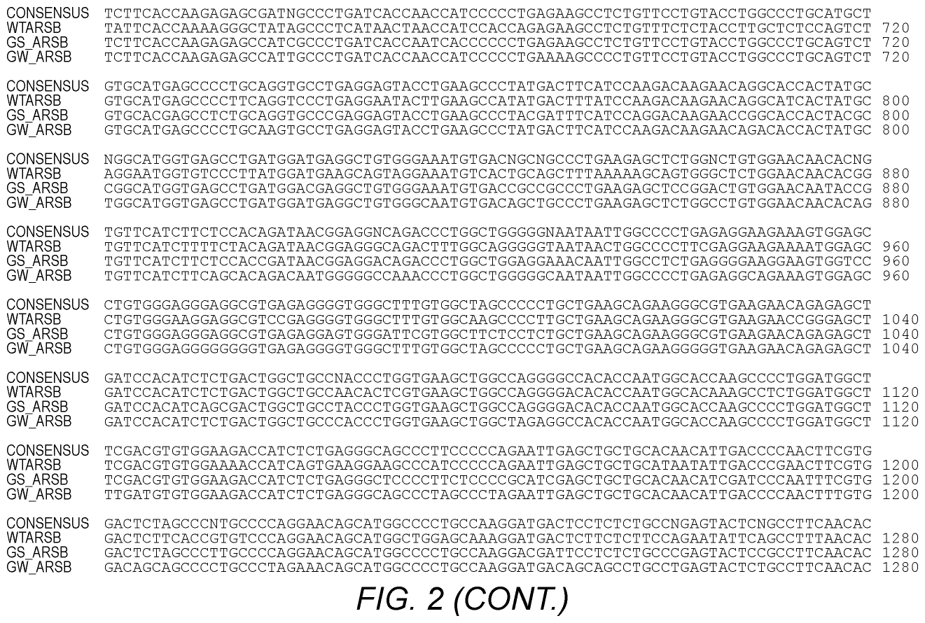

Two different codon-optimized versions of the ARSB open reading frame (GW and GS) were prepared (FIG. 1). A sequence comparison of GW and GS with wild-type ARSB is shown in FIG. 2. In vitro experiments were carried out including a quantitative comparison of expression of each open reading frame linked to the EF1α promoter in 293 cells. FIG. 3 shows the results of western blot analysis and cellular expression demonstrating the increased expression from the GS ORF relative to the GW and wild-type ORFs. While the overall enzyme activity from the two codon-optimized ORFs was somewhat comparable (FIG. 4), GS was selected for further studies as it has one fewer alternative ORF and is likely safer.

The GS ORF (opt-ArsB) was cloned into an AAV vector production plasmid. The vector was validated internally for titer, packaged genome integrity, and preparation contamination by Q-PCR, alkaline gel electrophoresis, and silver staining, respectively. This vector was aliquoted in low retention tubes and stored at −80° C. for use in in vivo experiments.

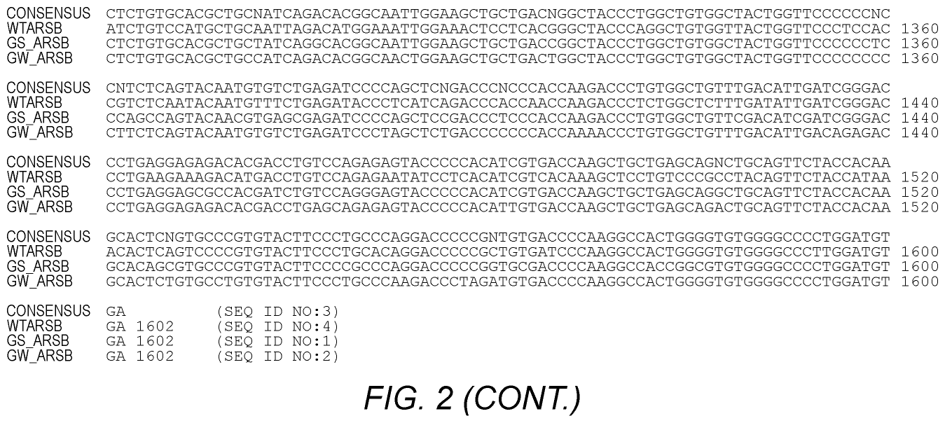

The AAV8-opt-ArsB vectors were administered to human cadaver corneas ex vivo. For these experiments, the vector (5×10⁹ vg) or saline was injected into the cornea stroma and the organs were cultured for 6 days thereafter. Protein lysate was prepared from the injected tissue. Vector injected cornea demonstrated elevation of arylsulfatase activity at that early time point post-injection demonstrating functionality (FIG. 5).

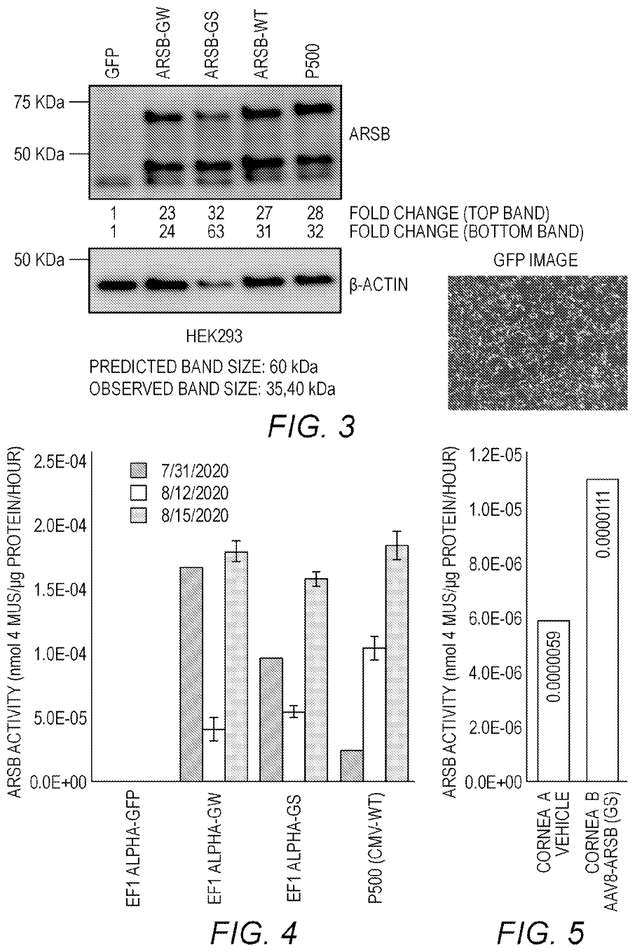

A feline model of MPS VI was used for in vivo studies (FIG. 6). MPS VI ArsB+/−and ArsB−/−kittens from a heterozygous breeding scheme were used. The model has an autosomal recessive single point mutation, a base substitution at codon 476 in the 4S-encoding gene that results in a leucine to proline change (L476P). Clinical evaluations were performed on these animals during weeks of acclimatization prior to injections using a subjective clinical scoring system and objective optical coherence topography (OCT) to determine central corneal thickness (CCT). Fundus imaging and electroretinography was also performed at monthly intervals. Clinically, storage disease was noted as diffuse corneal opacity only in the homozygote which progressed bilaterally over time. Fundus examinations were normal in both the homozygote and heterozygote litter mate. The homozygote in both corneas was thinner in CCT by OCT.

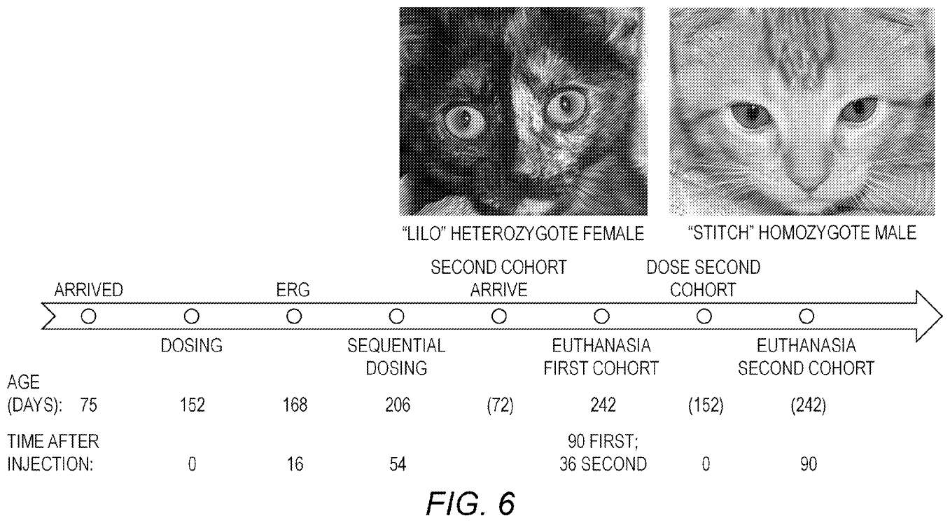

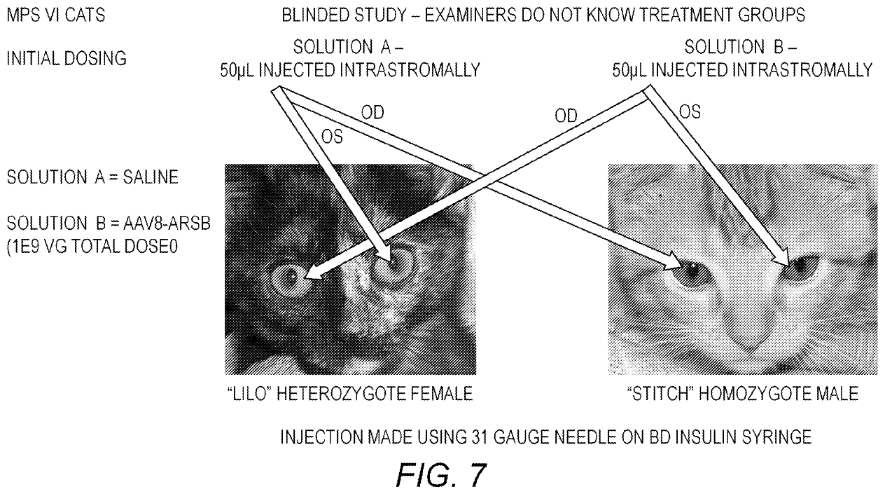

For the in vivo study the vehicle control (saline) or AAV8-opt-ArsB vector at a relatively low titer of 1×10⁹ vg were administered in 50 μl total volume per cornea (FIG. 7). The injections were well tolerated. Immediate cludiness was seen at the time of injection but cleared by 24 hours. No evidence of inflammation or immune response wa seen before or after injection in the homozygote or the heterozygote. (FIG. 8). These data demonstrated that the MPS VI feline corneas recover normally in the presence of pre-exisiting storage disease.

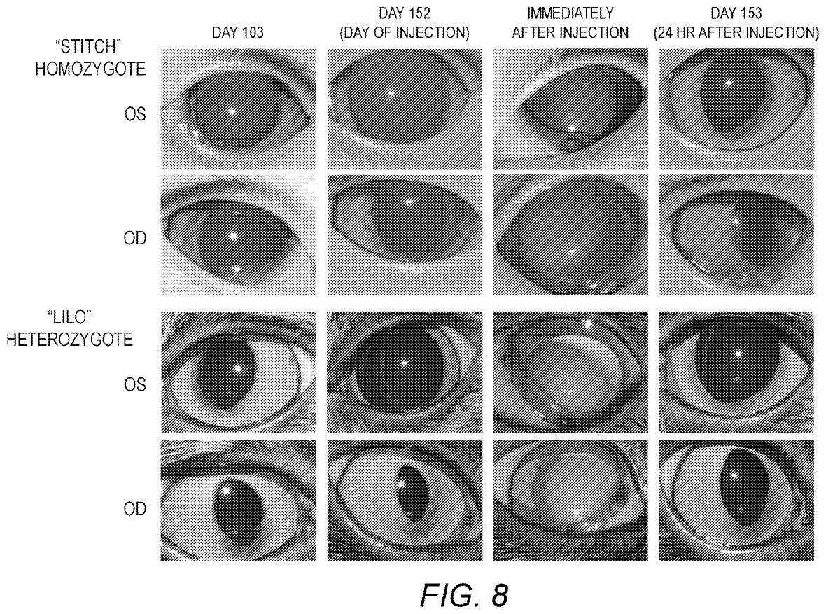

On day 21 post-injection phenotypic reversal was apparent in a single cornea of the ArsB−/− feline noted as a reduction in diffuse granularity (FIG. 9). The vehicle-injected cornea had a grit texture indicative of storage disease while the vector-injected cornea showed 85% storage disease clearance and normal dilation. This storage disease reversal progressed over time to clinical normalcy in approximately 90% of the cornea treated with vector. Corneal opacity was noted in the corneal limbal region which appeared to not be progressing in severity and might have been lessening over time. In the contralateral cornea of this animal injected with vehicle, diffuse corneal opacity progressed in severity over time.

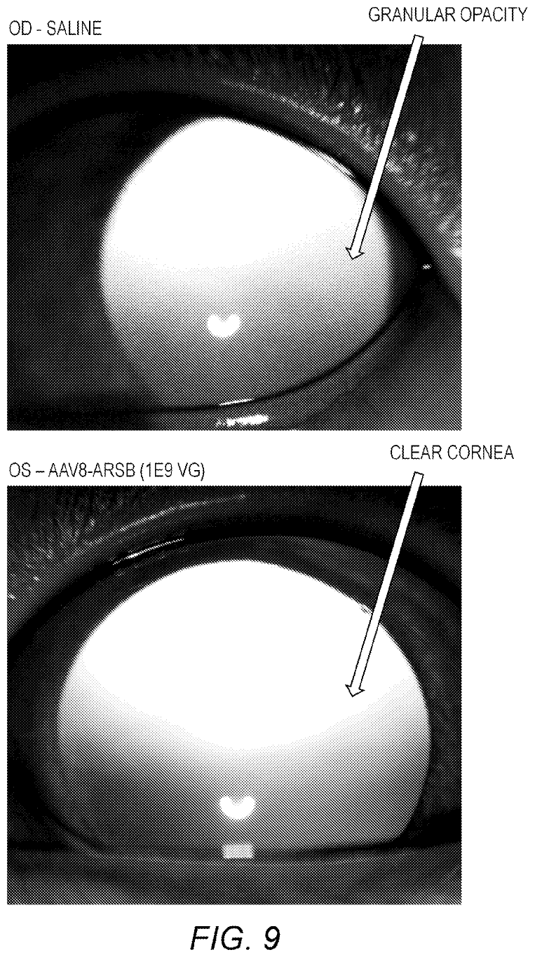

Following the success of the initial injection, AAV8-opt-ArsB vector was injected into the contralateral eye of the homozygote 54 days later to assess the effect of a second treatment. The second injection caused phenotypic reversal, demonstrating that repeat treatments in the same subject are possible (FIG. 10).

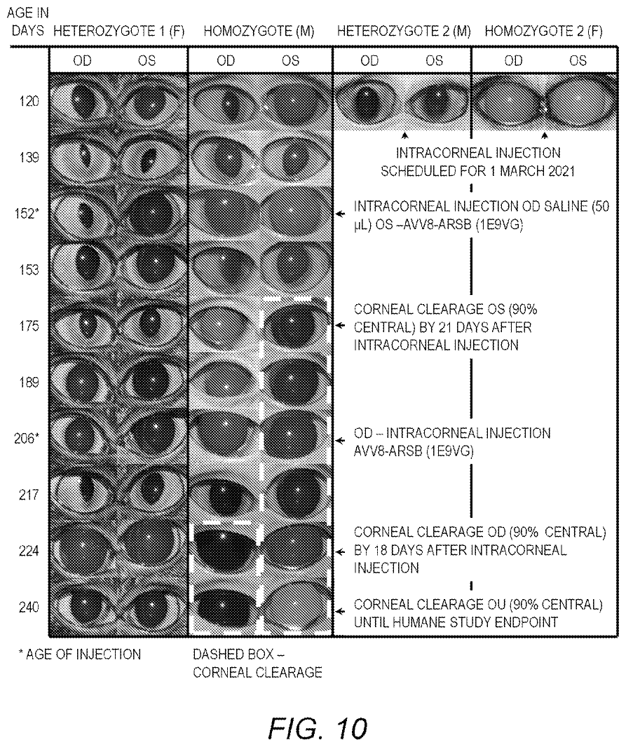

Cumulative inflammatory scores were determined. Ocular scores include density (1) and area of corneal opacity (4). Therefore, untreated MPS VI cats had a score of 5 prior to treatment. Heterozygote cats had a score of 0. A mild increase of inflammatory scores was observed at day 1 after injections (see Day 153) (FIG. 11). No other inflammatory changes were observed.

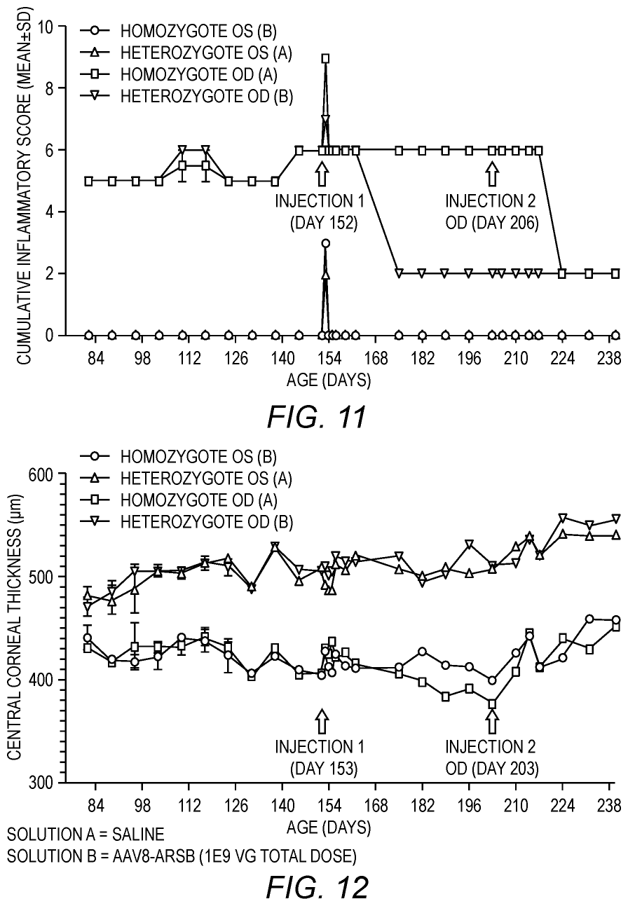

An analysis of corneal thickness changes shows that MPS VI corneas are thinner than wild-types corneas, similar to humans with MPS VI (FIG. 12). AAV8-opt-ArsB reversed the trend of reduced CCT.

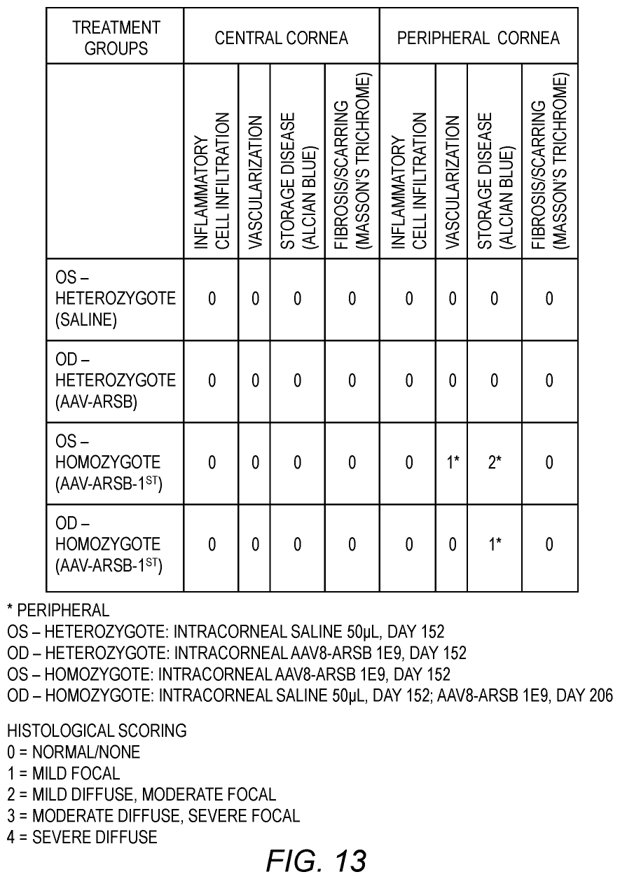

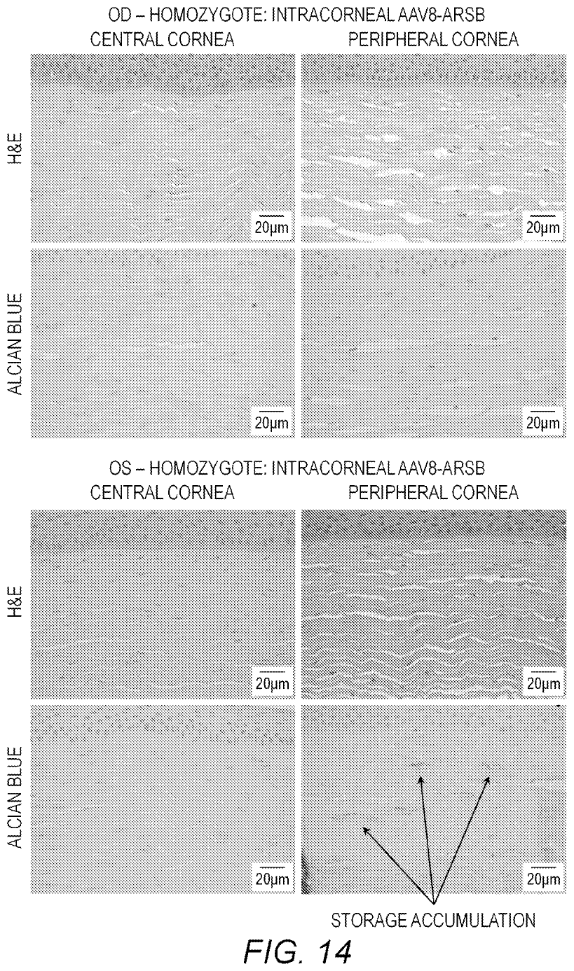

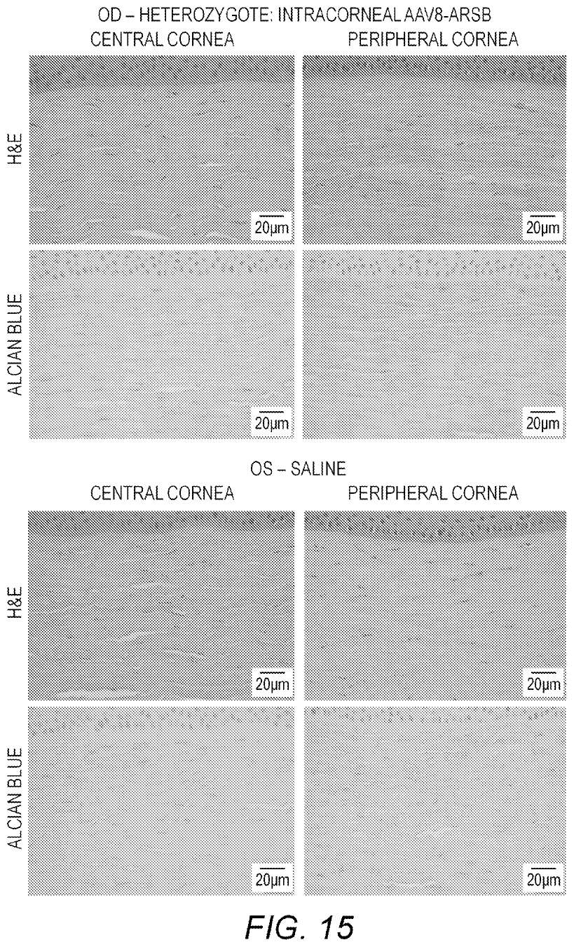

Corneal histopathology was performed following the study (FIGS. 13-15). No pathology was observed in the central cornea of treated eyes. Mild vascularization and storage disease was seen in the peripheral cornea.

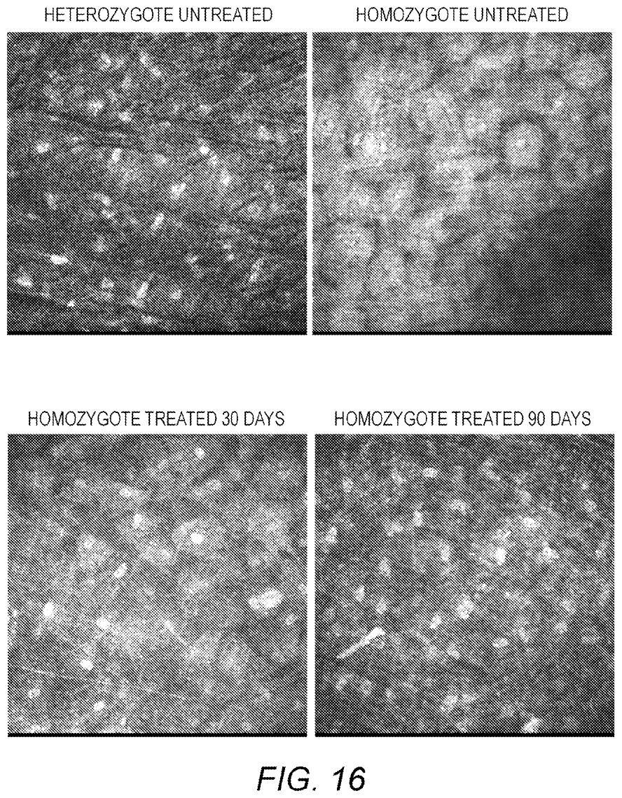

MPS VI −/− (symptomatic) feline cornea was dosed with AAV8-optArsB (intrastromal injection, 1×10⁹ vg per cornea in 50 μl final volume) and the posterior stroma was analyzed by in vivo microscopy at the indicated times post-injection. The results demonstrate progressive restoration of collagen/cellular structures compared to untreated corneas (heterozygous is asymptomatic) (FIG. 16).

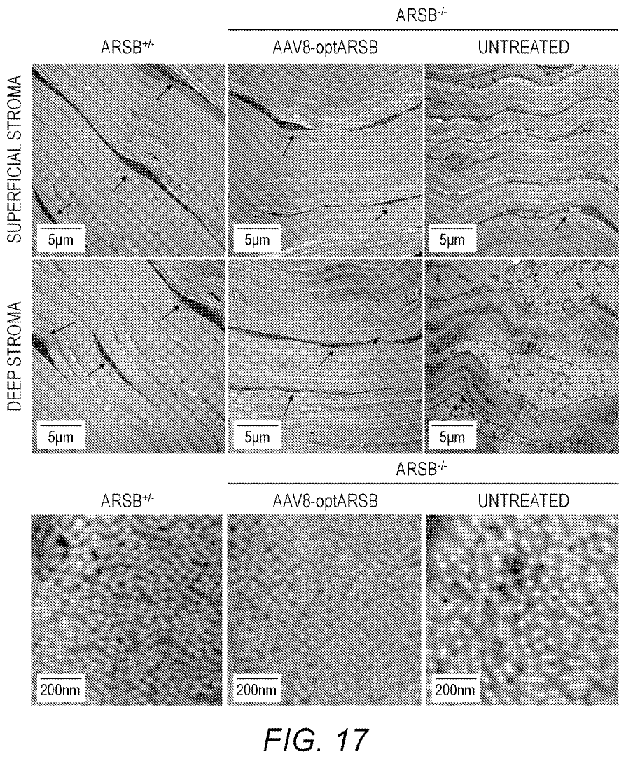

MPS VI −/− (symptomatic) feline cornea was dosed with AAV8-optArsB (intrastromal injection, 1×10⁹ vg per cornea in 50 μl final volume) and the corneas were analyzed by electron microscopy 6 months following the injection. The results demonstrate restoration of normal collagen alignment (upper panel) and fiber number (lower panel) in corneas treated with AAV8-optArsB (FIG. 17).

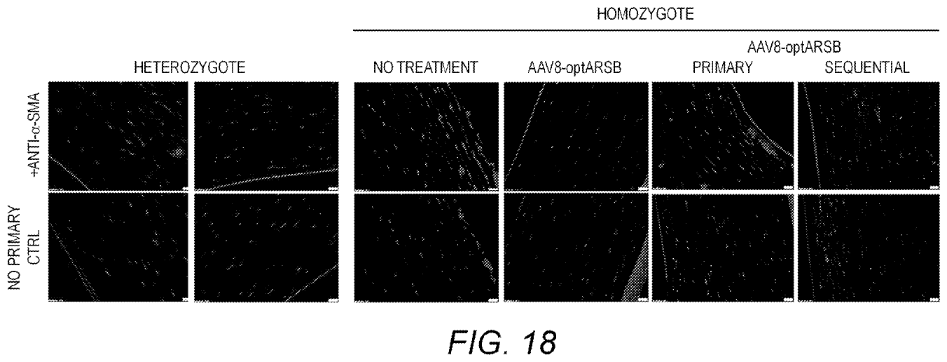

MPS VI −/− (symptomatic) feline cornea was dosed with AAV8-optArsB (intrastromal injection, 1×10⁹ vg per cornea in 50 μl final volume) and the corneas were analyzed by histological staining using an alpha smooth muscle antibody (indicator of corneal fibrosis). Decreased aSMA actin staining was observed in MPS VI corneas that received AAV8-optArsB (FIG. 18). Sequential dosing means the contralateral comea (initially not dosed) was injected with AAV8-opt-ArsB 6 weeks following the initial dose.

The foregoing is illustrative of the present invention, and is not to be construed as limiting thereof. The invention is defined by the following claims, with equivalents of the claims to be included therein.

1. A recombinant nucleic acid comprising a sequence encoding human arylsulfatase B (ARSB), wherein the nucleotide sequence has been codon-optimized for expression in human cells.

2. The recombinant nucleic acid of claim 1, comprising a nucleotide sequence at least 90% identical to SEQ ID NO:1 or SEQ ID NO:2.

3. The recombinant nucleic acid of claim 1, comprising the nucleotide sequence of SEQ ID NO:1 or SEQ ID NO:2.

4. A vector comprising the nucleic acid of claim 1.

5. The vector of claim 4, which is a viral vector.

6. The vector of claim 5, which is an adeno-associated virus (AAV) vector genome.

7. The vector of claim 4, wherein the nucleic acid is operably linked to a constitutive promoter.

8. The vector of claim 7, wherein the promoter is elongation factor 1α.

9. A cell in vitro comprising the vector of claim 4.

10. (canceled)

11. An AAV particle comprising the AAV vector genome of claim 6.

12. A method of producing a recombinant AAV particle comprising an AAV capsid, the method comprising:
providing a cell in vitro with AAV Cap and AAV Rep coding sequences, the AAV vector genome of claim 6, and helper functions for generating a productive AAV infection; and
allowing assembly of the recombinant AAV particle comprising the AAV capsid and encapsidating the AAV vector genome.

13. (canceled)

14. The AAV particle of claim 11, wherein the AAV particle is an AAV2, AAV8, or AAV9 particle.

15. The AAV particle of claim 11, wherein the AAV particle is a chimeric AAV8/AAV9 particle.

16. A pharmaceutical composition comprising the AAV particle of claim 11 and a pharmaceutically acceptable carrier.

17. (canceled)

18. A method of delivering ARSB to the eye of a subject, comprising administering to the eye of the subject an effective amount of a vector or polynucleotide that expresses ARSB or an ARSB polypeptide, thereby delivering ARSB to the eye of the subject.

19. (canceled)

20. A method of treating, slowing the progression of, or delaying the onset of a mucopolysaccharidosis VI (MPS VI)-associated ocular manifestation in a subject in need thereof, comprising administering to the eye of the subject a therapeutically effective amount of a vector or polynucleotide that expresses ARSB or an ARSB polypeptide, thereby treating, slowing the progression of, or delaying the onset of the MPS VI-associated ocular manifestation in the subject.

21. The method of claim 20, wherein the ocular manifestation is corneal clouding, glaucoma, optic nerve compression or degeneration, retinal degeneration, dilation issues, poor muscle control, or any combination thereof.

22-24. (canceled)

25. The method of claim 20, wherein the vector is an AAV particle.

26. (canceled)

27. The method of claim 20, wherein the vector, polynucleotide, or polypeptide is administered to the eye by intrastromal, topical, intracameral, intravitreal, subconjunctival, suprachoroidal, subtenon, retrobulbar, or subretinal administration.

28-29. (canceled)

30. The method of claim 20, wherein the subject is a human subject.

31-33. (canceled)
